(19) United States
(12) Patent Application Publication
(10) Pub. No.: US 2023/0076248 A1
(43) Pub. Date: Mar. 9, 2023

(54) METHODS AND KITS FOR CHARACTERIZING AND IDENTIFYING AUTISM SPECTRUM DISORDER

(71) Applicant: CELL-EL THERAPEUTICS LTD., Alon Shvut (IL)

(72) Inventors: Beni GESUNDHEIT, Alon Shvut (IL); Philip David ZISMAN, Mevasseret Tzion (IL); Ronald ELLIS, Jerusalem (IL); Ouriel FAKTOR, Rehovot (IL)

(21) Appl. No.: 17/796,712

(22) PCT Filed: Feb. 1, 2021
(86) PCT No.: PCT/IL2021/050113

§ 371 Date: Aug. 1, 2022

Related U.S. Application Data
(60) Provisional application No. 62/969,089, filed on Feb. 2, 2020.

(51) Int. Cl. G01N 33/68; G16H 50/30; G16H 50/70

(52) U.S. Cl. CPC G01N 33/6893; G16H 50/30; G16H 50/70; G01N 2800/28

(57) ABSTRACT

Provided herein are methods and kits for characterizing and for identifying autism and autism spectrum disorders.

### FIELD OF THE INVENTION

Provided herein are methods and kits for identifying autism and autism spectrum disorders (ASD).

### BACKGROUND OF THE INVENTION

ASD is a group of heterogeneous neurodevelopmental disorders presenting in early childhood with a prevalence of 0.7-2.6/100 subjects. ASD is generally detected in the second or third year of life, with final diagnosis typically obtained during the third or fourth year. Psychological treatment is considered to be most beneficial when initiated early in life, preferably during the second to fourth year of life, given that treatment tends to be less effective with age and ineffective after the age of seven or eight years. Furthermore, neuro-psychological tests, such as ADOS, are highly subjective and not always reliable for establishing early diagnosis of ASD, since accurate communication with very young children is challenging. Other diagnostic tests, such as the Childhood Autism Rating Scale (CARS), Communication and Symbolic Behaviour Scales (CSBS) and Social Responsiveness Scale 2 (SRS2), are not widely used and are not relied upon to the extent that ADOS is used.

U.S. Pat. No. 10,041,954 discloses the use of IL-6, IL-β and TNFα as biomarkers for diagnosing psychiatric/neurological disorder, such as, schizophrenia or autism, in adults.

U.S. Pat. No. 7,604,948 discloses the use of complement factor H-related protein (FHR1) alone, or in combination with other polypeptides, such as, TNFα, for diagnosing autism.

There is an unmet need for reliable, reproducible, and objective diagnostic markers and assays for identifying, in young children, ASD or susceptibility to ASD.

### SUMMARY OF THE INVENTION

There are provided methods for characterizing ASD, biomarkers and sets of biomarkers characterizing ASD, methods and biomarkers for diagnosing ASD, and kits for detecting ASD.

As described herein, the statistical analyses applied herein provide improved sensitivity, specificity, negative predictive value, positive predictive value, and/or overall accuracy for diagnosing ASD or risk of developing ASD.

To date, investigations for biomarkers in blood whose levels might correlate with risk for, or development of, ASD did not provide individual biomarker(s) or group(s)/panel(s) of biomarkers that identify, with a significant and reliable degree of certainty, ASD or risk for ASD in young children. Moreover, the etiology and neuropathology of ASD remain elusive; hence this information cannot be used for detecting ASD or susceptibility thereto. In addition, children with ASD do not constitute a homogeneous clinical group and many different pathologies show a similar constellation of behavioral symptoms that converge within the ASD spectrum.

Advantageously, disclosed herein are methods for characterizing ASD or susceptibility to ASD in biological samples, methods for identifying biomarkers characterizing ASD or susceptibility to ASD, panels of biomarkers which are unique to ASD or susceptibility to ASD and practical multi-biomarker diagnostic tests, such as decision trees (in the form of equations), for distinguishing ASD from control non-ASD subjects, with statistical significance and reproducibility. The data disclosed herein for individual biomarkers alone show the superiority of a multivariate model in that it generates a more balanced performance (e.g., Table 10).

Moreover, there are provided exemplary equations generated by multiple logistic regression (MLR)-based analyses, which demonstrate that the methods disclosed herein produce biomarkers and equations that correctly predict ASD cases and correctly identify typically developed (TD, meaning non-ASD normal children) cases. The particular equations disclose herein present an average accuracy of 10-fold cross-validation of 82±9%, an average sensitivity of 87±8%, and an average specificity of 77±14%.

In some embodiments, there is provided a method for identifying a plurality of protein biomarkers characterizing ASD or susceptibility to ASD, in a biological sample, the method comprising:
(a) obtaining a first group of biological samples from ASD subjects and a second group of biological samples from TD subjects;
(b) selecting a first set of proteins in said first group and a second set of proteins in said second group, wherein each of said first and second sets comprises a plurality of proteins;
(c) dividing the proteins in the first set into a first training subset and a first testing subset and the proteins in the second set into a second training subset and a second testing subset, wherein training subset : testing subset ratio in each of said first and second sets corresponds to first group : second group ratio;
(d) comparing the level of each protein in the first training subset to the level of said each protein in the second training subset and identifying protein biomarkers whose level in the first training subset is significantly different from its level in the second training subset; and
(e) selecting a plurality of protein biomarkers that are highly correlated and have lowest AIC value for constructing a multivariate logistic regression model, said plurality of protein biomarkers characterizes ASD, or susceptibility to ASD.

In some embodiments, the level of significance is calculated using Mann-Whitney test. In some embodiments, the method further comprises selecting a plurality of protein biomarkers having FDR-adjusted p-value<0.05 and FC>2, prior to step (e).

In some embodiments, said dividing is randomly dividing.

In some embodiments, step (b) further comprises filtering out proteins whose levels are below detectable level in more than 50% of each of said first and second groups.

In some embodiments, step (b) further comprises filtering out proteins whose levels are below detectable level in more than 60% of each of said first and second groups.

In some embodiments, the biological sample is derived from a subject of age between 1 year and 15 years.

In some embodiments, the biological sample is a blood sample, a serum sample or a plasma sample.

In some embodiments, the biological sample is a serum sample.

In some embodiments, the reference value corresponds to the level of said the plurality of protein biomarkers in biological samples derived from a population of TD subjects.

In some embodiments, the reference value for each protein biomarker in the plurality of protein biomarkers corresponds to the level of each said protein biomarker in biological samples derived from a population of TD subjects.

In some embodiments, the plurality of protein biomarkers is for diagnosing, predicting and prognosing ASD or susceptibility to ASD.

In some embodiments, the plurality of protein biomarkers is selected from the group consisting of the protein biomarkers listed in Table 4.

In some embodiments, the plurality of protein biomarkers comprises IL-17. In some embodiments, the plurality of protein biomarkers comprises at least one of IL-6 and IL-17. In some embodiments, the plurality of protein biomarkers comprises IL-6 and IL-17. In some embodiments, the plurality of protein biomarkers comprises at least one of IL-6, IL-10 and IL-17. In some embodiments, the plurality of protein biomarkers comprises at least one of IL-6, IL-9 and IL-17. In some embodiments, the plurality of protein biomarkers comprises at least one of IL-8, SR-A1 and IL-17. In some embodiments, the plurality of protein biomarkers is consisting of IL-6, IL-9 and IL-17. In some embodiments, the plurality of protein biomarkers is consisting of IL-8, SR-A1 and IL-17. In some embodiments, the plurality of protein biomarkers is selected from the group consisting of: IL-8, GM-CSF, IL-17, IL-10, IL-1ra, IL-6, IFN-γ, IL-12p70, G-CSF, IL-1a, IL-15 and AFP. In some embodiments, the plurality of protein biomarkers is selected from the group consisting of: GM-CSF, IL-1ra, AFP, IL-8, IL-15, IL-17, G-CSF and IL-6. In some embodiments, the plurality of protein biomarkers is selected from the group consisting of: G-CSF, GM-CSF, IL-6, IL-8, IL-15, IL-17 and AFP

In some embodiments, the plurality of protein biomarkers is selected from the group consisting of the protein biomarkers listed in Table 9. In some embodiments, the plurality of protein biomarkers is selected from the group consisting of G-CSF, IL-12p70, IL-9, IL-1b, IL-1ra, IL-17, IL-8, IL-6, IL-10, GM-CSF and IFN-γ. In some embodiments, the plurality of protein biomarkers is selected from the group consisting of CTNF, G-CSF, IL-12p70, IL-9, IL-1b, IL-1ra, Thrombospondin-2, IL-1a, IL-17, IL-8, IL-6, IL-10, GM-CSF and IFN-γ. In some embodiments, the plurality of protein biomarkers is selected from the group consisting of G-CSF, IL-12p70, IL-9, IL-1b, IL-1ra, IL-17, IL-8, IL-6, IL-10, GM-CSF, IFN-γ, BMPR-II, Common-beta-chain, Kremen-2, Desmoglein-2, NTB-A, MIG, IL-17R, aminopeptidase-LRAP and SR-A1. In some embodiments, the plurality of protein biomarkers is selected from the group consisting of G-CSF, IL-9, IL-1b, IL-1ra, IL-17, IL-8, IL-6, BMPR-II, Common-beta-chain, Kremen-2, Desmoglein-2, MIG, IL-17R, aminopeptidase-LRAP and SR-A1.

In some embodiments, there is provided a method for identifying a panel of protein biomarkers characterizing ASD or susceptibility to ASD, in a biological sample, the method comprising:
(a) obtaining a first group of biological samples from ASD subjects and a second group of biological samples from TD subjects;
(b) selecting a first set of proteins in said first group and a second set of proteins in said second group;
(c) dividing the proteins in the first and second sets into a plurality of folds, while maintaining, in each fold, first set: second set ratio similar to the first group: second group ratio;
(d) dividing each fold into first training subset and corresponding first testing subset, wherein the number of proteins in the first training set is larger than the number of proteins in the corresponding testing subset; and
(e) subjecting the training subset in each fold to multiple logistic regression, thereby identifying a panel of protein biomarkers characterizing ASD, or susceptibility to ASD.

In some embodiments, subjecting the training subset in each fold to multiple logistic regression, comprises obtaining an equation corresponding to each fold, the parameters of which comprise (i) a normalized level of each protein biomarker in a plurality of protein biomarkers from the panel of protein biomarkers and (ii) numerical coefficient corresponding to each protein biomarker in the plurality of protein biomarkers, wherein a result of an equation above a cutoff value indicates ASD or susceptibility to ASD.

In some embodiments, the plurality of folds comprises at least 5 folds.

In some embodiments, the number of proteins in the first training subset is at least two times larger than the number of proteins in the corresponding testing subset.

In some embodiments, the number of proteins in the first training subset is at least three times larger than the number of proteins in the corresponding testing subset.

In some embodiments, the biological sample is derived from a subject of age between 1 year to 15 years. In some embodiments, the biological sample is a blood sample, a serum sample or a plasma sample.

In some embodiments, the panel of protein biomarkers comprises a plurality of proteins selected from the group consisting of the protein biomarkers listed in Table 14. In some embodiments, the panel of protein biomarkers comprises a plurality of proteins selected from the group consisting of TNF-α, RBP4, SR-A1, IL-17, aFGF, IFN-γ, IL-10, IL-4, IL-6, IL-1a, procalcitonin, TC-PTP, TFPI, Kallikrein_1, Carboxypeptidase_A2, LIGHT, Semaphorin_7A, IL-8 and IL-9.

In some embodiments, the panel of protein biomarkers comprises at least one of TNF-α, IL-17, IL-10, IFN-γand aFGF. In some embodiments, the panel of protein biomarkers comprises TNF-α, IL-17, IL-10, IFN-γand aFGF. In some embodiments, the panel of protein biomarkers comprises at least IL-17, IL-10 and IL-6. In some embodiments, the panel of protein biomarkers further comprises at least one of RBP4, SR-Al, IL-4, IL-6, IL-1a, procalcitonin, TC-PTP, TFPI, Kallikrein_1, Carboxypeptidase_A2, LIGHT, Semaphorin_7A, IL-8 and IL-9.

In some embodiments, there is provided a kit for identifying a subject having ASD or susceptibility to ASD, the kit comprising: (a) means for measuring the level of a plurality of biomarker proteins selected from Tables 3, 4, 5, 6, 9 or 14 in a biological sample obtained from a subject; (b) a predetermined logistic regression model equation for the plurality of biomarkers and a cutoff value; and (c) means for obtaining a numerical value for the predetermined logistic regression model equation for the plurality of biomarker proteins, wherein a numerical value (Yi) above said cutoff value identifies said subject as having ASD or susceptibility to ASD.

In some embodiments, there is provided a kit for identifying ASD or susceptibility to ASD, the kit comprising: (a) means for measuring the level of a plurality of biomarker proteins selected from Tables 3, 4, 5, 6, 9 or 14 in a biological sample; (b) a predetermined logistic regression model equation for the plurality of biomarkers and a cutoff value; and (c) means for obtaining a numerical value for the predetermined logistic regression model equation for the plurality of biomarker proteins, wherein a numerical value (Yi) above said cutoff value identifies ASD or susceptibility to ASD.

In some embodiments, the means is a set of reagents configured to measure the levels of each protein biomarker in the plurality of protein biomarkers. In some embodiments, the reagents are binding molecules. In some embodiments, the binding molecules are antibodies.

In some embodiments, the plurality of biomarker proteins is selected from Table 3. In some embodiments, the plurality of biomarker proteins is selected from Table 4. In some embodiments, the plurality of biomarker proteins is selected from Table 5. In some embodiments, the plurality of biomarker proteins is selected from Table 6. In some embodiments, the plurality of biomarker proteins is selected from Table 9. In some embodiments, the plurality of biomarker proteins is selected from Table 14.

In some embodiments, the plurality of biomarker proteins comprises at least three biomarker proteins. In some embodiments, the at least three biomarker proteins comprise IL-6, IL-10 and IL-17.

In some embodiments, there is provided a method for diagnosing ex-vivo ASD or susceptibility to ASD, the method comprising:
(a) providing a predetermined logistic regression model equation for a plurality of biomarkers and a cutoff value, wherein the plurality of biomarker proteins is selected from Tables 3, 4, 5, 6, 9 or 14;
(b) determining, in a biological sample obtained from a subject, the level of each protein biomarker in the plurality of biomarker proteins; and
(c) incorporating the level of each protein biomarker in the predetermined logistic regression model equation, thereby obtaining a numerical value,
   wherein a numerical value above said cutoff value identifies said subject as having ASD or susceptibility to ASD.

In some embodiments, the plurality of biomarker proteins is selected from Table 5. In some embodiments, the plurality of biomarker proteins is selected from Table 6. In some embodiments, the plurality of biomarker proteins is selected from Table 9. In some embodiments, the plurality of biomarker proteins is selected from Table 14.

In some embodiments, the plurality of protein biomarkers comprises IL-17. In some embodiments, the plurality of protein biomarkers further comprises at least one protein selected from the group consisting of: IL-6, IL-8, IL-9, IL-10, G-CSF and GM-CSF.

In some embodiments, the plurality of protein biomarkers comprises at least three protein biomarkers. In some embodiments, the at least three biomarker proteins comprises at least one protein selected from the group consisting of: IL-17, IL-6 and IL-10. In some embodiments, the at least three biomarker proteins comprises IL-17, IL-6 and IL-10.

Other objects, features and advantages of the present invention will become clear from the following description, examples and drawings.

### BRIEF DESCRIPTION OF THE DRAWINGS

FIGS. 1A to 1G represent Receiver Operating Characteristic (ROC) graphs of seven (7) individual biomarkers: G-CSF (Granulocyte colony-stimulating factor; 1A), GM-CSF (Granulocyte-macrophage colony-stimulating factor; 1B), IL-6 (IL-6; 1C), IL-8 (1D), IL-15 (1E), IL-17 (1F) and AFP (Alpha-Fetoprotein; 1G), respectively.

FIG. 2 represents ROC curves plotted for the univariate models and the multivariate model, corresponding to Tables 7 and 8.

FIG. 3A represents hierarchical clustering based on correlation matrix for the 12 selected biomarkers shown in Table 5.

FIGS. 3B and 3C represent hierarchical clustering based on correlation (r<0.7) for repetitions A and B, respectively, the values of which are summarized in Table 9.

FIGS. 4A and 4B represent ROC and performance values obtained for each of repetition analysis A and B, respectively, the values of which are summarized in Tables 11 and 12.

### DETAILED DESCRIPTION

Provided herein are biomarkers for ASD. Further provided herein are methods for characterizing ASD and methods and kits for diagnosing ASD in a biological sample.

The term “biomarker” as used herein collectively refers to a single protein biomarker or a plurality of proteins, or protein biomarkers, which distinguish ASD and/or the risk or likelihood to developing ASD, in young children from normal, healthy, non-diseased, or TD population.

The terms “typically developing” or TD refer to subjects that are not afflicted with ASD or are not susceptible to ASD, also referred to as normal, healthy or non-diseased.

According to some embodiments, there is provided a method for characterizing ASD or susceptibility to ASD, in a biological sample.

The terms “method for characterizing ASD” and “method for identifying a plurality of protein biomarkers characterizing ASD” are interchangeable.

According to some embodiments, there is provided a method for identifying a plurality of protein biomarkers characterizing ASD or susceptibility to ASD, in a biological sample, the method comprising:
a) obtaining a first group of biological samples from ASD subjects and a second group of biological samples from TD subjects;
b) selecting a first set of proteins in said first group and a second set of proteins in said second group, wherein each of said first and second sets comprises a plurality of proteins;
c) dividing the proteins in the first set into a first training subset and a first testing subset and the proteins in the second set into a second training subset and a second testing subset, wherein training subset : testing subset ratio in each of said first and second sets corresponds to first group : second group ratio;
d) comparing the level of each protein in the first training subset to the level of said each protein in the second training subset and identifying a plurality of protein biomarkers whose level in the first training subset is significantly different from its level in the second training subset; and
e) selecting a plurality of protein biomarkers that are highly correlated and have lowest AIC value for constructing a multivariate logistic regression model, said plurality of protein biomarkers characterizes ASD, or susceptibility to ASD.

According to some embodiments, the biological sample is a sample obtained from a subject.

According to some embodiments, the first set of proteins comprises at least 100 proteins, at least 150 proteins, at least 200 proteins, at least 250 proteins, at least 300 proteins, or at least 350 proteins. Each possibility represents a separate embodiment.

According to some embodiments, the second set of proteins comprises at least 100 proteins, at least 150 proteins, at least 200 proteins, at least 250 proteins, at least 300 proteins, or at least 350 proteins. Each possibility represents a separate embodiment.

The terms “subject” and “patient” as used herein are interchangeable and refer to a human. A “patient” includes, but is not limited to, humans who are receiving medical care or persons, specifically, children, with no defined illness being investigated for signs of ASD.

The terms “sample” and “biological sample” refer to a sample that may be obtained from a subject. Preferred samples are body fluid samples.

The term “body fluid sample” as used herein refers to a sample of body fluid obtained for the purpose of diagnosis, classification or evaluation of a subject of interest, such as a patient. Preferred body fluid samples include blood, serum, plasma, cerebrospinal fluid, urine, saliva, sputum, and pleural effusions. In addition, one skilled in the art would realize that certain body fluid samples would be more readily analyzed following a fractionation or purification procedure, e.g., separation of whole blood into serum and plasma components.

The terms “diagnosing” and “diagnosis” as used herein refer to methods by which the skilled artisan can estimate and/or determine the probability (“a likelihood”) of whether a patient has ASD or is likely to develop, or be susceptible to the development, of ASD. In the case of the present invention, “diagnosis” includes using the results of an assay or analysis to help arrive at a diagnosis (i.e., the occurrence or nonoccurrence) of ASD for the subject from whom a sample was obtained and assayed. Since many biomarkers are indicative of multiple conditions, the skilled clinician does not use biomarker results in an informational vacuum, but rather test results are used together with other clinical indices to arrive at a diagnosis. Thus, a measured biomarker level on one side of a predetermined diagnostic threshold indicates a greater likelihood of the occurrence of ASD in the subject relative to a measured level on the other side of the predetermined diagnostic threshold.

The term “plurality” as used herein refers to at least two, more than 1, or two or more. According to some embodiments, the plurality of protein biomarkers selected in step (e) comprises at least three protein biomarkers. According to some embodiments, the plurality of protein biomarkers selected in step (e) comprises at least four protein biomarkers. According to some embodiments, the plurality of protein biomarkers selected in step (e) comprises at least five protein biomarkers. According to some embodiments, the plurality of protein biomarkers selected in step (e) comprises at least six protein biomarkers. According to some embodiments, the plurality of protein biomarkers selected in step (e) comprises at least seven protein biomarkers.

According to some embodiments, the subject is a child. According to some embodiments, the subject is a child within the age range of 15 y.o. to 1 y.o. According to some embodiments, the subject is a child within the age range of 15 y.o. to 2 y.o. According to some embodiments, the subject is a child within the age range of 15 y.o. to 3 y.o. According to some embodiments, the subject is a child within the age range of 14 y.o. to 3 y.o. According to some embodiments, the subject is a child within the age range of 14 y.o. to 2 y.o. According to some embodiments, the subject is a child within the age range of 13 y.o. to 2 y.o. According to some embodiments, the subject is a child within the age range of 13 y.o. to 3 y.o. According to some embodiments, the subject is a child within the age range of 12 y.o. to 2 y.o. According to some embodiments, the subject is a child within the age range of 11 y.o. to 2 y.o. According to some embodiments, the subject is a child within the age range of 10 y.o. to 2 y.o. According to some embodiments, the subject is a child within the age range of 9 y.o. to 2 y.o.

The term “significantly different” refers to p value<0.05. According to some embodiments, the level of significance is based on any suitable statistical method known in the art for establishing statistical significance, e.g., Mann-Whitney test and False Discovery Rate (FDR).

The term “highly correlated” is interchangeable with multi-colinearity and is a statistical phenomenon in which predictor variables in a logistic regression model are highly correlated. In order to evaluate the association between two or more variables (proteins) correlation test are used, these include, but are not limited to, Pearson's correlation, Spearman correlation and Kendall correlation, among others.

In some embodiments, selecting a plurality of protein biomarkers that have lowest AIC value comprises performing logistic regression on a plurality of protein biomarkers and selecting a plurality of protein biomarkers that have lowest AIC value.

According to some embodiments, the protein samples of each set are randomly divided to a “training subset” and a “testing subset”, such that in each subset the ratio between TD and ASD in the biological samples is preserved. For example, when starting by deriving biological samples from a population of 40% TD and 60% ASD, then the corresponding samples in each training and testing subset are about 40% TD and 60% ASD, respectively.

According to some embodiments, following identification of a first set of proteins in the first group, i.e., in the group derived from ASD subjects, and a second set of proteins in the second group, i.e., in the group derived from TD subjects, the method further comprises filtering out proteins whose levels are below detectable level in more than 40%, 45%, 50%, 55%, 60% or 65% of each of said first and second groups. Each possibility represents a separate embodiment.

The terms “threshold”, “cut-off” and “cutoff” as used herein are interchangeable and refer to value(s) distinguishing ASD from TD based on the technology disclosed herein.

According to some embodiments, the threshold value is a value which is most suitable for the purpose of the claimed method, namely, distinguishing ASD from TD. This value may be, for example, a statistical average obtained by measuring the level of each protein biomarker in a plurality of biological samples derived from a population of TD subjects and calculating the corresponding statistical average. According to some embodiments, the threshold value is obtained from logistic regression analysis applied for characterizing ASD, or susceptibility to ASD.

According to some embodiments, the biomarker is a polypeptide or a protein.

The terms “protein” and “polypeptide”, as used herein, are interchangeable.

According to some embodiments, the biomarker comprises a plurality of proteins. According to some embodiments, the plurality of biomarker proteins are cytokines and/or chemokines.

Several cytokines and chemokines have been shown to be associated with ASD. For example, increased serum levels of IL-12p40 were shown in children with autism. Other proteins such as Epidermal growth factor (EGF), where binding thereof to EGFR results in cellular proliferation, differentiation and survival, were shown to be overexpressed in children with ASD. Another protein, CD134 (also known as OX40L), was found to be upregulated in ASD.

Furthermore, elevated levels of growth-related hormones, such as Insulin-like growth factor-binding proteins IGFBP-6 and IGFBP-3 as well as each of Nestin, VEGF (Vascular endothelial growth factor) and VEGFR2, were found in adults with ASD. The presence of maternal thyroid peroxidase antibody (TPOab) increased risk for ASD by nearly 80%. Carbonic Anhydrase Type 2 (CA2) deficiency syndrome was shown to correlate with ASD. Some connection between Ra1A (Ras family small GTP binding protein) or RBP4 (Retinol binding protein 4) with ASD has been shown.

The correlation between each of TNF-α (also termed herein TNFa or TNF-a), GM-CSF, IL-6R or IL-17 with ASD shown to date is contradictory. Some studies showed association with ASD, while other studies presented the opposite. It is worth noting that IL-17 induces the production of various cytokines, such as, IL-6, G-CSF, GM-CSF, IL-β, TGF-β and TNF-α, chemokines (including IL-8, GRO-α (Growth-regulated oncogene), and MCP-1 (Monocyte chemoattractant protein 1) and prostaglandins (e.g., PGE2).

CD99 was not shown to be associated with ASD. However, the immune function genes CD99L2, JARID2 (jumonji and AT-rich interaction domain containing 2) and TPO (thyroperoxidase) showed association with ASD. Moreover, none of the following proteins were shown to be related to ASD: Prolargin (Proline-arginine-rich end leucine-rich repeat protein; PRELP), Aminopeptidase P2, Carboxypeptidase A2, Fetuin-A, HCC-4, Matrilin-3, Osteoactivin, Siglec-5, IL-16, TFPI (Tissue factor pathway inhibitor), Fc receptor-like protein 2 (FCRL2) and SR-Al (Scavenger receptor class A member 1).

According to some embodiments, the plurality of protein biomarkers selected from the protein biomarkers listed in Tables 3 and 4. According to some embodiments, the plurality of protein biomarkers is selected from the protein biomarkers listed in Table 3. According to some embodiments, the plurality of protein biomarkers is selected from the protein biomarkers listed in Table 4.

According to some embodiments, the plurality of protein biomarkers comprises at least two protein biomarkers selected from the protein biomarkers listed in Table 5.

According to some embodiments, the plurality of protein biomarkers is selected from the group consisting of: IL-8, GM-CSF, IL-17, IL-10, IL-1ra, IL-6, IFN-γ, IL-12p70, G-CSF, IL-1a, IL-15 and AFP. According to some embodiments, the plurality of protein biomarkers is selected from the group consisting of: GM-CSF, IL-1ra, AFP, IL-8, IL-15, IL-17, G-CSF and IL-6. According to some embodiments, the plurality of protein biomarkers is selected from the group consisting of: G-CSF, GM-CSF, IL-6, IL-8, IL-15, IL-17 and AFP.

According to some embodiments, the plurality of protein biomarkers is selected from the group consisting of the protein biomarkers listed in Table 9. According to some embodiments, the plurality of protein biomarkers is selected from the group consisting of G-CSF, IL-12p70, IL-9, IL-1b, IL-1ra, IL-17, IL-8, IL-6, IL-10, GM-CSF and IFN-γ. According to some embodiments, the plurality of protein biomarkers is selected from the group consisting of CNTF (Ciliary neurotrophic factor), G-CSF, IL-12p70, IL-9, IL-1b, IL-1ra, Thrombospondin-2, IL-1a, IL-17, IL-8, IL-6, IL-10, GM-CSF and IFNγ. According to some embodiments, the plurality of protein biomarkers is selected from the group consisting of G-CSF, IL-12p70, IL-9, IL-1b, IL-1ra, IL-17, IL-8, IL-6, IL-10, GM-CSF, IFNγ, BMPR-II (Bone morphogenetic protein receptor type-2), Common-beta-chain, Kremen-2, Desmoglein-2, NTB-A (NK-T-B-antigen), MIG (Monokine induced by IFNγ), IL-17R, aminopeptidase-LRAP and SR-A1. According to some embodiments, the plurality of protein biomarkers is selected from the group consisting of G-CSF, IL-9, IL-1b, IL-1ra, IL-17, IL-8, IL-6, BMPR-II, Common-beta-chain, Kremen-2, Desmoglein-2, MIG, IL-17R, aminopeptidase-LRAP and SR-A1.

In some aspects, there are provided methods, systems, and strategies in the form of statistical analyses, specifically, multivariate analysis for identifying a combination of protein biomarkers (also denoted herein “marker” or “biomarker”) associated with ASD, which are useful for diagnosing ASD and the risk or likelihood to develop ASD, in young children. To date, due to the lack of reliable objective test for ASD, many children do not receive a final diagnosis until much older. In fact, some children are not diagnosed until they are adolescents or adults. This delay means that children with ASD might not get the early help they need. Non-diagnosed children with ASD might have difficulties, as adolescents and young adults, in developing and maintaining friendships, communicating with peers and adults, or understanding what behaviors are expected in school or on the job. They may come to the attention of healthcare providers due to co-occurring conditions such as attention-deficit/hyperactivity disorder, obsessive compulsive disorder, anxiety or depression, or conduct disorder. Thus, diagnosing children with ASD as early as possible is highly important as diagnosed children can receive the services and support they need to reach their full potential.

The statistical analyses applied herein advantageously provide improved sensitivity, specificity, negative predictive value, positive predictive value, and/or overall accuracy for diagnosing ASD or risk of developing ASD.

The terms “associate”, “relate” and “correlate” as used herein in reference to the use of biomarkers refer to comparing the presence or amount of the biomarker(s) in a biological sample, such as a biological sample obtained from a patient to a reference standard. The reference standard may be an ASD reference, e.g., the presence or amount of said biomarker(s) in persons with, or known to be at risk of developing, ASD; or a TD reference, such as the presence or amount of said biomarker(s) in persons known to be free of ASD. Often, this takes the form of comparing an assay result in the form of a biomarker concentration to a predetermined threshold selected to be indicative of the occurrence or non-occurrence of ASD or the likelihood of some future outcome associated with ASD.

Selecting a diagnostic threshold involves consideration of the probability of disease and distribution of true and false diagnoses at different test thresholds, among other considerations.

Suitable thresholds may be determined in a variety of ways predominantly derived from statistical analyses. For example, one recommended diagnostic threshold for the diagnosis of ASD is the 97.5th percentile of the concentration seen in a normal (TD) population.

Population studies may also be used to select a decision threshold. ROC analysis is often used to select a threshold able to best distinguish a “diseased” subpopulation from a “non-diseased” subpopulation. A false-positive finding in this case occurs when the person tests positive, but actually does not have the disease. A false-negative finding, on the other hand, occurs when the person tests negative, suggesting they are healthy, when they actually do have the disease or are susceptible to it. To draw a ROC curve, the true positive rate (TPR) and false positive rate (FPR) are determined as the decision threshold is varied continuously. Since TPR is equivalent with sensitivity and FPR is equal to 1-specificity, the ROC graph is sometimes called the sensitivity vs (1-specificity) plot. A perfect test will have an area under the ROC curve of 1.0; a random test will have an area of 0.5. A threshold is selected to provide an acceptable level of specificity and sensitivity.

The terms “statistical analysis”, “statistical algorithm” and “statistical process” are interchangeable and include any of a variety of statistical methods and models used to determine relationships between variables. In the present disclosure, the variables are the presence and relative level of a plurality of markers/proteins of interest which together form a biomarker for ASD. Any number of markers can be analyzed using a statistical analysis described herein. For example, the presence or level of 1, 2, 3, 4, 5, 6, 7, 8, 9, 10, 11, 12, 13, 14, 15, 16, 17, 18, 19, 20, 25, 30, 35, 40, 45, 50, 55, 60, or more markers can be included in a statistical analysis.

Determination of the biomarker disclosed herein was carried out using various statistical analyses. However, any of a variety of statistical methods, models and algorithms, including those described below, may be used.

Identification of the diagnostic biomarker profile (cluster, combination) disclosed herein initiated with quantitative determination of the levels of numerous cytokines in ASD and TD biological samples.

Any method for quantitative determination of protein levels known in the art may be applied, such as, but not limited to, immunochemical techniques, e.g., immunoblot, immunoassay, multiplex immunoassay, enzyme-linked immunosorbent assay (ELISA), radioimmunoassay, immunoradiometric assay, fluorescent immunoassay, chemiluminescent immunoassay and immunonephelometry. Preferred methods are those enabling the quantification of numerous proteins in one experiment at high accuracy, efficiency, specificity and sensitivity.

Upon identification of proteins in the sample, removal of markers, the level of which was not available or not detectable, was performed. Removal of non-relevant markers may be performed by any suitable statistical method, including, but not limited to, heatmap analysis, volcano plots, and principal component analysis (PCA).

In some embodiments, the statistical analysis is a multivariate analysis. In some embodiments, the statistical analysis comprises a multivariate logistic regression model. In other embodiments, the statistical analysis comprises a stepwise logistic regression using the multivariate logistic regression model. In some embodiments, a plurality of protein biomarkers corresponding to a multivariate logistic regression model having the lowest/smallest Akaike Information Criterion (AIC) value are selected for constructing the model.

In some embodiments, the plurality of protein biomarkers having the lowest AIC comprise IL-6 and IL-17.

In some embodiments, the plurality of protein biomarkers comprises IL-17. In some embodiments, the plurality of protein biomarkers comprises at least one of IL-6 and IL-17. In some embodiments, the plurality of protein biomarkers comprises IL-6 and IL-17.

A number of multiple logistic regression (MLR) techniques were applied herein to identify relevant biomarker combinations for ASD as further detailed below.

According to some embodiments, there is provided a method for identifying a panel of protein biomarkers characterizing ASD or susceptibility to ASD, in a biological sample, wherein the panel of biomarkers is determined by multiple logistic regression applied on proteins identified in biological samples derived from ASD and TD subjects, the method comprising:
a) obtaining a first group of biological samples from ASD subjects and a second group of biological samples from TD subjects;
b) selecting a first set of proteins in said first group and a second set of proteins in said second group;
c) dividing the proteins in the first and second sets into a plurality of folds, while maintaining, in each fold, a first set : second set ratio similar to the first group : second group ratio;
d) dividing each fold into first training subset and corresponding first testing subset, wherein the number of proteins in the first training set is larger than the number of proteins in the corresponding testing subset; and
e) subjecting the training subset in each fold to MLR, thereby identifying a panel of protein biomarkers characterizing ASD, or susceptibility to ASD.

In a dataset, a training set is used to build up a model, while a testing (or validation) set is applied to validate the model built. Data points in the training set are excluded from the testing (validation) set. The proportion of training to testing sets may vary, where a proportion of 50%:50% produces a different precision from a 10%:90%. In general, the bigger the dataset to train is better.

The term “panel” as used herein refers to group, set, combination or the like, of protein biomarkers characterizing ASD or susceptibility to ASD. A panel of protein biomarkers includes two or more protein biomarkers.

According to some embodiments, the plurality of folds comprises at least 5 folds, at least 6 folds, at least 7 folds, at least 8 folds, at least 9 folds, at least 10 folds or at least 12 folds. Each possibility represents a separate embodiment.

According to some embodiments, the number of proteins in the first training set is at least two times larger than the number of proteins in the corresponding testing subset. According to some embodiments, the number of proteins in the first training set is at least three times larger than the number of proteins in the corresponding testing subset.

According to some embodiments, the method further comprises applying zero filtering and feature correlation clustering filtering on the training subset in each fold, prior to said subjecting. According to some embodiments, the method further comprises the step of filtering out proteins whose levels are substantially zero, prior to said subjecting step.

According to some embodiments, the panel of biomarkers comprises at least two proteins selected from the proteins listed in Table 14. According to some embodiments, the panel of biomarkers comprises at least three proteins selected from the proteins listed in Table 14. According to some embodiments, the panel of biomarkers comprises at least four proteins selected from the proteins listed in Table 14. According to some embodiments, the panel of biomarkers comprises at least five proteins selected from the proteins listed in Table 14. According to some embodiments, the panel of biomarkers comprises at least six proteins selected from the proteins listed in Table 14. According to some embodiments, the panel of biomarkers comprises at least seven proteins selected from the proteins listed in Table 14.

In some embodiments, the panel of biomarkers comprises a plurality of biomarkers that occurred in at least 80% of the plurality of folds. In some embodiments, the panel of biomarkers comprises a plurality of biomarkers that occurred in at least 90% of the plurality of folds. In some embodiments, the panel of biomarkers comprises a plurality of biomarkers that occurred in each and every fold of the plurality of folds.

According to some embodiments, the panel of biomarkers is consisting of the proteins listed in Table 14.

According to some embodiments, the panel of protein biomarkers comprises at least two proteins selected from IL-17, aFGF, IFN-y, IL-10 and TNFα. According to some embodiments, the panel of protein biomarkers comprises at least three proteins selected from IL-17, aFGF, IFN-γ, IL-10 and TNFα. According to some embodiments, the panel of protein biomarkers comprises at least four proteins selected from IL-17, aFGF, IFN-γ, IL-10 and TNFα. According to some embodiments, the panel of protein biomarkers comprises IL-17, aFGF, IFN-γ, IL-10 and TNFα.

According to some embodiments, the panel of protein biomarkers comprises IL-17, aFGF, IFN-γ, IL-10 and TNFα and at least one of RBP4, TFPI, SR-A1, IL4-Ra, IL-6, IL-1a, procalcitonin, TC-PTP, Kllikrein_1, carboxypeptidase_A2, LIGHT, semaphoring-7A, IL-8 and IL-9. According to some embodiments, the panel of protein biomarkers comprises IL-17, aFGF, IFN-γ, IL-10 and TNFα and at least two of RBP4, TFPI, SR-A1, IL4-Ra, IL-6, IL-1a, procalcitonin, TC-PTP, Kllikrein_1, carboxypeptidase_A2, LIGHT, semaphoring-7A, IL-8 and IL-9. According to some embodiments, the panel of protein biomarkers comprises IL-17, aFGF, IFN-γ, IL-10 and TNFα and at least three of RBP4, TFPI, SR-A1, IL4-Ra, IL-6, IL-1a, procalcitonin, TC-PTP, Kllikrein_1, carboxypeptidase_A2, LIGHT, semaphoring-7A, IL-8 and IL-9.

According to some embodiments, the panel of protein biomarkers comprises TNFα, RBP4, TFPI, SR-A1, IL-17, aFGF, IFN-γ, IL-10, IL-4Ra, IL-6, IL-1a, procalcitonin, TC-PTP, Kallikrein_1, carboxypeptidase_A2, LIGHT, semaphorin-7A, IL-8 and IL-9.

Several protein biomarkers were found to distinguish ASD from TD, independently of the statistical analyses or the initial database (first or second/expanded), namely, IL-17, IL-6, IL-9, IL-1a, IL-8, IL-10, IFNγ and SR-A1.

Thus, according to some embodiments, the panel of protein biomarkers comprises a plurality of proteins selected from the group consisting of IL-17, IL-6, IL-9, IL-1a, IL-8, IL-10, IFNg and SR-A1 .

The statistical process comprises MLR, which is a method that attempts to best fit the coefficients of a logistic formula constructed from the values of a small set (training set) of chosen features, each with its own factor. The heart of the method is choosing those features that best fit the predictions of this method in the training set to actual cases. It is essential to test this method on new data (testing set), since the algorithm can randomly find features that split the data provided to it in a way that fits the classes; since the test data were not used to define the splitting method, testing the resulting equations on data not used to build them gives a realistic estimate of their performance with new data.

The MLR analysis may include a 10-fold cross-validation, an approach in which 90% of the data is used for training every cycle and 10% for testing, replacing the cases used for testing 10 times and thus getting 10 equations and 10 performance statistics. If random trees are generated, the accuracy obtained with the 10 test sets should be the same as guessing and the trees generated for the different folds should be unrelated. To reject the hypothesis that the equations have random numbers for performance, they need to perform better than guessing in the test sets or the 10 equations should be similar to each other.

According to some embodiments, the subjecting step refers to subjecting the training subset in each fold MLR, thereby obtaining at least one logistic regression model equation and a corresponding threshold (also termed “cutoff value”) for characterizing ASD, or susceptibility to ASD.

According to some embodiments, the value of each protein biomarker in the logistic regression model equation corresponds to its amount, expression level, concentration, and the like, in a sample. According to some embodiments, the value of each protein biomarker in the logistic regression model equation corresponds to its normalized value (calculated from the measured values) in a sample.

According to some embodiments, the value of each protein biomarker in logistic regression formulas (i)-(iii) corresponds to its amount in a sample. According to some embodiments, the value of each protein biomarker in logistic regression formulas (iv)-(xiii) corresponds to its normalized value (calculated from the measured values) in a sample.

The term “about” as used herein can allow for a degree of variability in a value or range, for example, within 20%, within 15%, within 10%, within 5%, or within 1% of a stated value, limit or range of values.

In some embodiments, the statistical process further includes measuring test accuracy to determine the effectiveness of a given biomarker. These measures include sensitivity and specificity, predictive values, likelihood ratios, diagnostic odds ratios, and ROC curve areas. The area under the curve (“AUC”) of a ROC plot is equal to the probability that a classifier will rank a randomly chosen positive instance higher than a randomly chosen negative one. The area under the ROC curve may be thought of as equivalent to the Mann-Whitney U test, which tests for the median difference between scores obtained in the two groups considered if the groups are of continuous data, or to the Wilcoxon test of ranks.

In some embodiments, there is provided a kit for identifying a subject having ASD or susceptibility to ASD, the kit comprising: (a) means for measuring the level of a plurality of biomarker proteins selected from Tables 3, 4, 5, 6, 9 or 14 in a biological sample obtained from a subject; and (b) at least one predetermined logistic regression model equation for the plurality of biomarkers and at least one corresponding cutoff value; (c) means for calculating the at least one predetermined logistic regression model equation for the plurality of biomarker proteins and obtaining a numerical value, wherein a numerical value above said cutoff value identifies said subject as having ASD or susceptibility to ASD.

In some embodiments, there is provided a kit for identifying ASD or susceptibility to ASD, the kit comprising: (a) means for measuring the level of a plurality of biomarker proteins selected from Tables 3, 4, 5, 6, 9 or 14 in a biological sample; (b) a predetermined logistic regression model equation for the plurality of biomarkers and a cutoff value; and (c) means for obtaining a numerical value for the predetermined logistic regression model equation for the plurality of biomarker proteins, wherein a numerical value (Yi) above said cutoff value identifies ASD or susceptibility to ASD.

In some embodiments, the kit comprises a receptacle containing the means for measuring the level of the plurality of biomarker proteins selected from Tables 3, 4, 5, 6, 9 or 14.

In some embodiments, the kit comprises a storage device comprising the predetermined logistic regression model equation and the corresponding cutoff value for the plurality of biomarker proteins. According to some embodiments the storage device maybe a disc-on-key, a CD and the like. Alternatively, the kit may include instructions for downloading an app or for entering a website, and the like, and further instructions and/or codes (e.g. one or more passwords) required for obtaining the logistic regression model equation and the corresponding cutoff for the plurality of biomarker proteins.

According to some embodiments, the app and/or website enable to calculate the numerical value (Yi) for the logistic regression model equation and may further provide an output indicating ASD or susceptibility to ASD when the numerical value is above said cutoff value.

In some embodiments, the means is a set of reagents configured to measure the levels of each protein biomarker in the plurality of protein biomarkers. In some embodiments, the reagents are binding molecules. In some embodiments, the binding molecules are antibodies.

According to some embodiments, the means for measuring the level of said plurality of biomarker proteins comprises a plurality of antibodies suitable for quantitative analyses, such as, ELISA and protein immunoprecipitation combined with multiple reaction monitoring mass spectrometry (IP-MRM). Alternatively, the means for measuring the level of said plurality of biomarker proteins comprises a plurality of probes suitable for quantitative western blotting.

It is to be understood that for each panel/set/plurality of specific biomarkers selected from the protein biomarkers disclosed herein (listed in Tables 3, 4, 5, 6, 9 or 14) there is (i) a corresponding predetermined logistic regression model equation; and (ii) a cutoff value, based on which identification of ASD is carried out per the measured levels of the biomarkers in the selected panel. Accordingly, in some embodiments, the kit disclosed herein may be specific for a particular biomarker panel and hence may include a single predetermined logistic regression model equation and a corresponding cutoff value. Alternatively, in some embodiments, the kit disclosed herein may be specific for a particular biomarker panel and include more than one predetermined logistic regression model equation and a corresponding cutoff value for each equation. In some embodiments, the kit is configured for more than one biomarker panel and hence includes corresponding predetermined logistic regression model equation and a cutoff value for each biomarker panel.

The kit may be operatively associated with detection device, such as, an optical system, adapted to detect the reagents that bind to the protein biomarkers, and then evaluate the level of each protein biomarker. To this end, the reagents may be labeled, for example, may include a fluorescent tag.

In general, as used herein, a component that is “operatively associated with” one or more other components indicates that such components are directly connected to each other, in direct physical contact with each other without being connected or attached to each other, or are not directly connected to each other or in contact with each other, but are mechanically, electrically (including via electromagnetic signals transmitted through space), or fluidically interconnected (e.g., via channels such as tubing) so as to cause or enable the components so associated to perform their intended functionality.

In some embodiments, kit is operatively associated with a detection device, or a detector.

In some embodiments, the means for calculating comprise a processor. The processor can be programmed, using microcode or software, to perform the calculations. The processor may be operatively associated with a variety of components including, but not limited to, user interface and a detection device as detailed above. The processor may be a component within a computer implemented system, a server, and the like, adapted to carry out the analytic steps detailed herein for the purpose of identifying a subject having ASD or susceptibility to ASD, based on the plurality of protein biomarkers.

In some embodiments, the means for calculating can be a computer software which receives as an input the level of each protein biomarker in a panel/list of protein biomarkers. In some embodiments, the computer software directs a computer processor to perform the calculation and the comparison to the cutoff value and accordingly to determine ASD or TD, per biological sample.

The computer software can include processor-executable instructions that are stored on a non-transitory computer readable medium. The computer software can also include stored data, such as, predetermined logistic regression model equation for the plurality of biomarkers and a cutoff value per equation (per a panel of protein biomarker). The computer readable medium can be a tangible computer readable medium, such as a compact disc (CD), magnetic storage, optical storage, random access memory (RAM), read only memory (ROM), or any other tangible medium.

In some embodiments, the user interface is configured to obtain input from a user, such as, a list of a plurality of protein biomarkers the level of which should be determined and then incorporated into a predetermined logistic regression model equation corresponding to the list of biomarker proteins. In some embodiments, the user interface is configured to provide the numeric value as an output of the calculation carried out by the processor. In some embodiments, the user interface is configured to provide an output of the calculation carried out by the processor in the form of “ASD” or “TD”, based on a comparison between the numeric value and the cutoff value.

In some embodiments, the predetermined logistic regression model equation is an equation or formula (e.g. a logit equation), determined using a multivariate model to predict whether a given sample belongs to the TD or ASD.

In some embodiments, the predetermined logistic regression model equation is generated by multiple logistic regression analyses. Exemplary equations generated by multiple logistic regression analyses are presented in Table 15.

In some embodiments, the plurality of biomarker proteins comprises biomarkers selected from Table 3, having a Pearson's correlation coefficient higher than 0.7 and low AIC value. In some embodiments, the plurality of biomarker proteins comprises biomarkers selected from Table 4, having a Pearson's correlation coefficient higher than 0.7 and low AIC value. In some embodiments, the plurality of biomarker proteins comprises biomarkers selected from Table 5, having a Pearson's correlation coefficient higher than 0.7 and low AIC value. In some embodiments, the plurality of biomarker proteins comprises biomarkers selected from Table 6, having a Pearson's correlation coefficient higher than 0.7 and low AIC value. In some embodiments, the plurality of biomarker proteins comprises biomarkers selected from Table 9, having a Pearson's correlation coefficient higher than 0.7 and low AIC value.

In some embodiments, the plurality of biomarker proteins comprises biomarkers selected from Table 3, having a coefficient equal or higher than 0.8 in the logistic regression model equation. In some embodiments, the plurality of biomarker proteins comprises biomarkers selected from Table 4, having a coefficient equal or higher than 0.8 in the logistic regression model equation. In some embodiments, the plurality of biomarker proteins comprises biomarkers selected from Table 5, having a coefficient equal or higher than 0.8 in the logistic regression model equation. In some embodiments, the plurality of biomarker proteins comprises biomarkers selected from Table 6, having a coefficient equal or higher than 0.8 in the logistic regression model equation. In some embodiments, the plurality of biomarker proteins comprises biomarkers selected from Table 9, having a coefficient equal or higher than 0.8 in the logistic regression model equation.

In some embodiments, the plurality of biomarker proteins comprises at least three biomarker proteins. In some embodiments, the at least three biomarker proteins comprise IL-6, IL-10 and IL-17. In some embodiments, the plurality of biomarker proteins comprises at least four biomarker proteins. In some embodiments, the plurality of biomarker proteins comprises at least five biomarker proteins. In some embodiments, the plurality of biomarker proteins comprises at least six biomarker proteins. In some embodiments, the plurality of biomarker proteins comprises at least seven biomarker proteins. In some embodiments, the plurality of biomarker proteins comprises at least eight biomarker proteins.

It is to be understood that the kit, or its individual components, may represent an automated system (such as, a robotic system) or may be incorporated in an automated system that receives biological samples, determines the level of each biomarker in a specific plurality of biomarkers, determines for each biological sample the numerical value of a predetermined logistic regression model equation for the specific plurality (panel) of protein biomarkers, and then provides an output indicating ASD or TD.

In some embodiments, there is provided an automated system for identifying ASD or susceptibility to ASD, in a biological sample, the system comprises:
(a) a detector configured to measure the level of at least one plurality of biomarker proteins selected from Tables 3, 4, 5, 6, 9 or 14 in a biological sample obtained from a subject;
(b) a database comprising at least one predetermined logistic regression model equation for the at least one plurality of biomarkers, and a corresponding cutoff value; and
(c) at least one processor, in communication with the detector and the database, programmed to evaluate the at least one predetermined logistic regression model equation for the at least one plurality of biomarkers, based on the measured values obtained from the detector, produce a corresponding numerical value and output an indication of ASD or susceptibility to ASD when the numerical value is above said cutoff value.

In some embodiments, the automated system is configured to provide diagnostic output for more than one panel of protein biomarkers. In some embodiments, the system is configured (e.g. via the processor) to select for a selected panel of biomarkers, the corresponding logistic regression equation and cutoff.

The automated system may be connected to LAN networking environment through a network interface or adapter. When used in a WAN networking environment, the automated system may typically include a modem or other means for establishing communications over the WAN, such as the Internet.

In some embodiments, the database is an interactive database configured to be updated with logistic regression model equations and cutoff values corresponding to a plurality of panel of biomarkers, selected from the biomarkers in Tables 3, 4, 5, 6, 9 and 14. The database may be stored in a remote memory storage device associated with the automated system through the internet, Bluetooth, and the like, or via physical electronic wiring or communication (e.g. USB, disc-on-key, CD and the like).

According to some embodiments, the automated system is configured to identify biomarkers and sets of biomarkers which reliably distinguish ASD from TD, with high specificity and sensitivity, based on the current disclosure.

In some embodiments, the plurality of biomarker proteins is selected from Table 5. In some embodiments, the plurality of biomarker proteins is selected from Table 6. In some embodiments, the plurality of biomarker proteins is selected from Table 9. In some embodiments, the plurality of biomarker proteins is selected from Table 14. In some embodiments, the plurality of protein biomarkers is consisting of IL-17 and IL-6, said cutoff value is 0.072 and said predetermined logistic regression model equation is:

Yi=4.7−0.036*IL-6−0.03*IL-17

In some embodiments, the plurality of protein biomarkers is consisting of IL-17, IL-9 and IL-6, said cutoff value is 1.064 and said predetermined logistic regression model equation is:

Yi=5−0.012*IL-6−0.0885*IL-17−0.0005*IL-9

In some embodiments, the plurality of protein biomarkers is consisting of IL-8, SR-A1and IL-17, said cutoff value is 1.176 and said predetermined logistic regression model equation is:

Yi=4.76+0.015*IL-8−0.1*IL-17−0.001*SR-A1

In some embodiments, the plurality of protein biomarkers is consisting of IFNγ, IL-10, IL-17, TNFα, aFGF, IL-4Ra, IL-6, IL1a and RBP4, said cutoff value is 0.5 and said predetermined logistic regression model equation is:

P=exp(Yi)/(1+exp(Yi)

wherein Yi=−0.13*IFNγ−1.25*IL-10−0.84*IL-17+0.27*TNFα−1.70*aFGF+1.09*IL-4Ra−1.08*IL-6−0.31*IL-1a−0.66*RBP4−0.33

In some embodiments, the plurality of protein biomarkers is consisting of IFNγ, IL-10, IL-17, TNFα, aFGF, IL-4Ra, IL-6, IL1a and TFPT, said cutoff value is 0.5 and said predetermined logistic regression model equation is:

P=exp(Yi)/(1+exp(Yi)

wherein Yi=−0.22*IFNγ−1.09*IL-10−0.96*IL-17+0.32*TNFα−1.76*aFGF+1.42*IL-4Ra−1.07*IL-6−0.11*IL-1a−0.48*TFPI−0.34

In some embodiments, the plurality of protein biomarkers is consisting of IFNγ, IL-10, IL-17, TNFα, aFGF, IL-4Ra, IL-6, IL1a and TFPT, said cutoff value is 0.5 and said predetermined logistic regression model equation is:

P=exp(Yi)/(1+exp(Yi)

wherein Yi=−0.23*IFNγ−1.15*IL-10−1.03*IL-17+0.33*TNFα−1.59*aFGF+1.08*IL-4Ra-0.87*IL-6−0.11*IL-1a−0.56*TFPI−0.34

In some embodiments, the plurality of protein biomarkers is consisting of IFNγ, IL-10, IL-17, TNFα, aFGF, IL-4Ra, IL-6, IL1a and Kallikrein_1, said cutoff value is 0.5 and said predetermined logistic regression model equation is:

P=exp(Yi)/(1+exp(Yi)

wherein Yi=−0.08*IFNγ−1.00*IL-10−1.03*IL-17−0.13*TNFα−1.54*aFGF+1.026*IL-4Ra−0.81*IL-6−0.24*IL-1a−0.93*Kallikrein_1−0.49

In some embodiments, the plurality of protein biomarkers is consisting of IFNγ, IL-10, IL-17, TNFα, aFGF, LIGHT, IL-6, IL1a and Semaphorin_7A, said cutoff value is 0.5 and said predetermined logistic regression model equation is:

P=exp(Yi)/(1+exp(Yi)

wherein Yi=0.17*IFNγ−0.69*IL-10−1.16*IL-17−0.04*TNFα−2.14*aFGF+1.07*LIGHT−0.51*IL-6−0.45*IL-1a−0.67*Semaphorin_7A−0.68

In some embodiments, the plurality of protein biomarkers is consisting of IFNγ, IL-10, IL-17, TNFα, aFGF, IL-4Ra, IL-6, Procalcitonin and TFPI, said cutoff value is 0.5 and said predetermined logistic regression model equation is:

P=exp(Yi)/(1+exp(Yi)

wherein Yi=−0.16*IFNγ−1.19*IL-10−0.93*IL-17+0.11*TNFα−1.44*aFGF+0.92*IL-4Ra−1.14*IL-6+1.43*Procalcitonin−0.48*TFPI−0.22

In some embodiments, the plurality of protein biomarkers is consisting of IFNγ, IL-10, IL-17, TNFα, aFGF, IL-4Ra, IL-6, Procalcitonin and TCPTP, said cutoff value is 0.5 and said predetermined logistic regression model equation is:

P=exp(Yi)/(1+exp(Yi)

wherein Yi=−0.02*IFNγ−1.06*IL-10−0.98*IL-17+0.26*TNFα−1.14*aFGF+0.84*IL-4Ra−1.35*IL-6+1.27*Procalcitonin−0.66*TCPTP−0.17

In some embodiments, the plurality of protein biomarkers is consisting of IFNγ, IL-10, IL-17, TNFα, aFGF, IL-4Ra, IL-6, Procalcitonin and TCPTP, said cutoff value is 0.5 and said predetermined logistic regression model equation is:

P=exp(Yi)/(1+exp(Yi)

wherein Yi=−0.07*IFNγ−0.92*IL-10−0.89*IL-17−0.15*TNFα−1.17*aFGF+0.80*IL-4Ra−1.39*IL-6+1.44*Procalcitonin−0.81*TCPTP−0.26

In some embodiments, the plurality of protein biomarkers is consisting of IFNγ, IL-10, IL-17, TNFα, aFGF, IL-4Ra, Carboxypeptidase_A2, Procalcitonin and Kallikrein_1, said cutoff value is 0.5 and said predetermined logistic regression model equation is:

P=exp(Yi)/(1+exp(Yi)

wherein Yi=−0.36*IFNγ−1.25*IL-10−1.44*IL-17+0.12*TNFα−1.35*aFGF+0.85*IL-4Ra−1.05*Carboxypeptidase_A2+1.58*Procalcitonin−0.58*Kallikrein_1−0.28

In some embodiments, the plurality of protein biomarkers is consisting of IFNγ, IL-10, IL-17, TNFα, aFGF, IL-4Ra, IL-6, IL-1a and TFPI, said cutoff value is 0.5 and said predetermined logistic regression model equation is:

P=exp(Yi)/(1+exp(Yi)

wherein Yi=−0.23*IFNγ−1.15*IL-10−1.03*IL-17+0.33*TNFα−1.59*aFGF+1.08*IL-4Ra−0.87*IL-6−0.1*IL-1a−0.56 TFPI−0.34

In some embodiments, there is provided a method for diagnosing a subject having ASD or susceptibility to ASD, the method comprising:
(a) providing a predetermined logistic regression model equation for a plurality of biomarkers and a cutoff value, wherein the plurality of biomarker proteins is selected from Tables 3, 4, 5, 6, 9 or 14;
(b) determining, in a biological sample obtained from a subject, the level of each protein biomarker in the plurality of biomarker proteins; and
(c) incorporating the level of each protein biomarker in a predetermined logistic regression model equation generated for the plurality of biomarker proteins, thereby obtaining a numerical value,
   wherein a numerical value above said cutoff value identifies said subject as having ASD or susceptibility to ASD.

In some embodiments, the subject of age between 1 year to 15 years. In some embodiments, the biological sample is a blood sample, a serum sample or a plasma sample.

In some embodiments, the method is carried out ex-vivo. In some embodiments, the method for diagnosing ASD is a method for diagnosing ASD ex-vivo. In some embodiments, the method for diagnosing ASD is a method for diagnosing ASD in vitro.

In some embodiments, the plurality of biomarker proteins is selected from Table 5. In some embodiments, the plurality of biomarker proteins is selected from Table 6. In some embodiments, the plurality of biomarker proteins is selected from Table 9. In some embodiments, the plurality of biomarker proteins is selected from Table 14.

In some embodiments, there is provided a method for identifying ASD or susceptibility to ASD in a biological sample, the method comprising:
(a) providing a predetermined logistic regression model equation for a plurality of biomarkers and a cutoff value, wherein the plurality of biomarker proteins is selected from Tables 3, 4, 5, 6, 9 or 14;
(b) determining, in a biological sample the level of each protein biomarker in the plurality of biomarker proteins; and
(c) incorporating the level of each protein biomarker in a predetermined logistic regression model equation generated for the plurality of biomarker proteins, thereby obtaining a numerical value,
   wherein a numerical value above said cutoff value identifies ASD or susceptibility to ASD.

The term “incorporating the level of each protein biomarker in a predetermined logistic regression model equation” refers to inserting the value determined in step (b) or a normalized value corresponding thereto, into the equation, where applicable. Following said incorporating, calculation of the predetermined logistic regression model equation is carried out, resulting with a numerical value. The calculation may be performed manually, or via a suitable calculator, algorithm, processor, software and the like.

The predetermined logistic regression model equation is generated through multivariate analysis or MLR, for a plurality of pre-selected biomarker proteins which uniquely distinguish ASD from TD, as exemplified herein. The numerical value obtained from the calculation, from each biological sample, is then compared to the cutoff value corresponding to the equation, wherein, a numeric value higher than the cutoff value indicates that the subject has ASD or susceptibility to have ASD.

Combining assay results comprise the use of multivariate logistical regression, loglinear modeling, neural network analysis, n-of-m analysis, etc. This list is not meant to be limiting.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

### EXAMPLES

### Example 1

### Study Set Up and Sample Collection

Numerous (˜1,000) biomarkers were tested in sera obtained from ASD subjects and normal, or typically developing (TD), subjects. The goal of the study was to identify biomarkers with improved specificity and sensitivity relative to those of a single biomarker or even combinations of biomarkers. The study included a combination of statistical tests. MLR equations obtained herein provide practical and efficient multi-biomarker diagnostic tests, which can be applied using known detection methods, e.g., ELISA. In practice, each bioassay for a respective set of biomarkers is performed and then the equations lead the operator to a decision on whether the subject should be assigned to the ASD group or to the TD group.

Subjects (age 3-12 years) who met the inclusion criteria (Table 1) for the study were recruited and divided into two groups: ASD children and TD children.

TABLE 1
Inclusion and exclusion criteria

| Inclusion Criteria | | Exclusion Criteria | |
|---|---|---|---|
| ASD | TD | ASD | TD |
| Ages 3-12 | Ages | Seizure disorder | Seizure disorder |
| Clear | 3-12 | requiring ongoing | requiring ongoing |
| diagnosis | | pharmaceutical | pharmaceutical |
| of ASD | | intervention | intervention |
| based | | Mild infection | Mild infection |
| onDSM | | (cold/fever/antibiotics) | (cold/fever/antibiotics) |
| | | during the last month | during the last month |
| | | Serious infection | Serious infection |
| | | during the last 6 mo. | during the last 6 mo. |
| | | Use systemic steroids | Use systemic steroids |
| | | in the last 6 mo. | in the last 6 mo. |
| | | Cancer-treatment | Cancer-treatment |
| | | in the last 6 mo. | in the last 6 mo. |
| | | Siblings | Siblings |

Sera were collected according to standard protocols. Briefly, at least 5 mL whole blood were collected from each child in a single venepuncture, and a 1mL aliquot was sent for standard CBC analysis. The remaining blood was transferred to BD Vacationer Separator tubes (yellow cap) and mixed 6 times by inversion. Blood was allowed to clot in an upright position for 30-60 min at room temperature, then blood tubes were centrifuged for 10 min at 1,000g. After removing rubber stoppers, 0.5 mL serum from each tube was aliquoted into labelled and chilled plastic screwcap cryovials. The vials were immediately frozen on dry ice and transferred to −80° C. For transportation, boxes with vials aliquoted serum samples were placed on dry ice with a digital thermometer inside the dry ice box. Upon arrival to destination, data from the thermometer were reviewed by the receiver entity to verify proper conditions of sample transportation.

Multiplexed sandwich ELISA-based quantitative array platform was applied to determine the concentration of multiple cytokines simultaneously in each serum sample, where a pair of cytokine-specific antibodies were used for detection. This approach combines the advantages of high detection sensitivity and specificity of ELISA, high assay throughput, and the ability to rapidly assay up to 1,000 analytes with only a very small volume of serum (<0.1 mL). For quantification, array-specific cytokine standards of predetermined concentrations were applied to generate a quantitative standard curve for each cytokine. The level of each cytokine/biomarker was measured using the KiloPlex array (RayBiotech; a high-density multiplex platform that enables the quantification of 1,000 human cytokines in a single experiment). In total, two related databases were examined. A first database composed of 102 ASD samples (68% of total) and 43 TD samples (32% of total) and a second database, also termed hereinafter “expanded database” that included the 102 ASD samples and 43 TD samples from the first database and additional 54 TD samples, thereby forming a database composed of 102 ASD samples (52% of total) and 97 TD samples (48% of total). In addition, a positive control sample (designated ‘BG’) was run in each array on the KiloPlex array platform.

### Example 2

### Identification of Biomarker Combinations for ASD/TD Prediction Using 70:30 Validation Approach—Univariate Analysis

2.1. Exclusion of Biomarkers with Overall Very Low Expression Levels

The usual first step in the construction of a predictive model is the selection of a small set of relevant features (i.e., proteins) to be used in the model. The database for measured levels of 1,000 biomarkers, generated for 102 ASD samples and 43 TD samples, was filtered using the following approach: proteins whose levels were below detectable levels in >60% of the samples (both ASD and TD groups) were filtered out from further analyses; 103 biomarkers (Table 2) fulfilled these conditions.

TABLE 2
Rejected biomarkers (n = 103)

| Name | Name | Name | Name | Name | Name | Name |
|---|---|---|---|---|---|---|
| MCSF | bFGF | BMP-4 | BTC | IL-28A | IL-29 | I-TAC |
| LIF | BCAM | ErbB2 | GROa | MMP-2 | APRIL | ACE-2 |
| ANG-4 | BAFF | CXCL14 | GASP-2 | IL-17B R | LAG-3 | RANK |
| SOST | TRANCE | Troponin I | WISP-1 | Dkk-4 | EDA-A2 | FGF-6 |
| FGF-9 | Gas 1 | IGFBP-5 | IL-1 F6 | IL-17C | IL-10 Ra | Layilin |
| Leptin R | PSMA | SIGIRR | BMPR-IA | Cadherin-11 | Nectin-4 | Persephin |
| CEACAM-5 | Cystatin A | Pref-1 | ALK-1 | Desmoglein-1 | Cathepsin E | Presenilin 1 |
| TREM-2 | Activin RIIB | CD109 | CREG | Dectin-1 | Endoglycan | p27 |
| ULBP-3 | CLEC-2 | PD-L2 | CD5 | CD69 | CK19 | DDR1 |
| MIA | PEAR1 | PTH1R | Serpin A5 | CD36 | ADAMTSL-1 | BATF3 |
| Bora | CD2 | CD200 R1 | CHST3 | Cystatin SA | EXTL3 | FRS2 |
| HIF-1 beta | HSD17B1 | Kell | Olig2 | Pax3 | TAZ | CHMP2B |
| Contactin-3 | GRAP2 | NCK1 | NUDT5 | UNC5H3 | CA5A | EphB3 |
| Galanin | MEF2C | B7-H4 | Bcl-10 | Cyclophilin A | GDF-9 | GPR115 |
| KIR2DL3 | NKp80 | Syntaxin 4 | TAFA2 | TMEFF1 | | |

2.2 Division of the Biomarker Level Database Into Training and Testing Sets

To provide an unbiased evaluation of the predictive model, the samples were randomly divided to a training set and a testing set, with about 70% and 30%, respectively, of the samples in each set, such that in each set, the dataset ratio between TD (32%) and ASD (68%) was preserved. The predictive model was built on the training set, then evaluated on the testing set for validation.

2.3 Selection of Biomarkers with Significant Differential Levels in ASD and TD

For each of the proteins/biomarkers in the training set, a Mann-Whitney Test (M-W) was conducted to compare levels between the ASD and TD groups. This selection revealed 159 biomarkers (Table 3) that had a significant difference in levels between the ASD and TD groups (M-W p-value<0.05). Of this group, a subgroup of 36 biomarkers that had FDR-adjusted p-value<0.05, and FC>2 are listed in Table 4.

TABLE 3
Biomarkers (n = 159) with M-W p value < 0.05

| | | | Fold | | | | Fold |
|---|---|---|---|---|---|---|---|
| Protein | p-value < | FDR adj | Change | Protein | p-value < | FDR adj | Change |
| (biomarker) | 0.05 | p-value | (FC) | (biomarker) | 0.05 | p-value | (FC) |
| IL-6 | 7.88E−12 | 3.54E−09 | −2.85 | Syndecan.4 | 1.09E−02 | 1.20E−01 | −1.09 |
| IL-17 | 5.34E−11 | 1.26E−08 | −3.78 | CES2 | 1.10E−02 | 1.20E−01 | −2.28 |
| IL-12p70 | 5.59E−11 | 1.26E−08 | −2.40 | CD84 | 1.19E−02 | 1.27E−01 | −1.56 |
| IL-10 | 2.65E−10 | 4.77E−08 | −3.07 | FGF-19 | 1.24E−02 | 1.31E−01 | −1.70 |
| TNFa | 1.00E−09 | 1.50E−07 | −1.95 | Mer | 1.33E−02 | 1.39E−01 | −1.27 |
| GM-CSF | 1.45E−09 | 1.87E−07 | −6.31 | IL-31 | 1.35E−02 | 1.39E−01 | −1.51 |
| IL-8 | 1.89E−09 | 2.12E−07 | −6.48 | Cf10 | 1.36E−02 | 1.39E−01 | 1.34 |
| IFNg | 4.87E−09 | 4.86E−07 | −2.54 | Carboxypeptidase.A2 | 1.44E−02 | 1.45E−01 | −1.47 |
| IL-1a | 1.97E−08 | 1.68E−06 | −2.16 | MIG | 1.50E−02 | 1.47E−01 | −1.76 |
| TNFb | 2.05E−08 | 1.68E−06 | −1.86 | HAI.2 | 1.50E−02 | 1.47E−01 | −1.37 |
| IL-13 | 3.59E−08 | 2.69E−06 | −1.79 | IL-24 | 1.52E−02 | 1.47E−01 | −1.64 |
| IL-11 | 9.50E−08 | 6.56E−06 | −1.60 | Galectin.1 | 1.53E−02 | 1.47E−01 | −1.37 |
| IL-15 | 1.05E−07 | 6.63E−06 | −2.05 | Siglec-11 | 1.61E−02 | 1.54E−01 | −8.13 |
| RBP4 | 1.11E−07 | 6.63E−06 | −1.19 | AKR1C4_x000D | 1.66E−02 | 1.57E−01 | −2.15 |
| IL-4 | 1.50E−07 | 8.42E−06 | −1.71 | DSPG3 | 1.74E−02 | 1.63E−01 | −2.07 |
| IL-7 | 3.17E−07 | 1.67E−05 | −1.59 | CA19-9 | 1.78E−02 | 1.65E−01 | −1.26 |
| IL-5 | 1.16E−06 | 5.78E−05 | −1.63 | MDM2 | 1.81E−02 | 1.66E−01 | −1.60 |
| IL-16 | 2.32E−06 | 1.10E−04 | −1.78 | TSK | 1.85E−02 | 1.67E−01 | −1.14 |
| Fetuin-A | 3.15E−06 | 1.41E−04 | −1.36 | ADAM8 | 1.93E−02 | 1.74E−01 | −1.87 |
| VEGF-R2 | 6.54E−05 | 2.80E−03 | 1.26 | PRX2 | 1.97E−02 | 1.74E−01 | N/D |
| IL-1-R3 | 9.27E−05 | 3.79E−03 | −1.21 | PDGF-Rb | 1.98E−02 | 1.74E−01 | −1.49 |
| IL-6R | 1.55E−04 | 6.04E−03 | −1.12 | LRIG3 | 2.05E−02 | 1.78E−01 | N/D |
| SR-AI | 1.63E−04 | 6.12E−03 | −1.84 | Integrin.alpha.5 | 2.06E−02 | 1.78E−01 | −1.30 |
| BLC | 2.22E−04 | 7.96E−03 | −1.67 | SDF-1b | 2.14E−02 | 1.83E−01 | 1.55 |
| CF-XIV | 3.32E−04 | 1.15E−02 | −1.92 | Glypican.5 | 2.16E−02 | 1.83E−01 | −1.57 |
| IL-1ra | 3.79E−04 | 1.25E−02 | −2.94 | RELT | 2.22E−02 | 1.86E−01 | −1.17 |
| G-CSF | 3.91E−04 | 1.25E−02 | −2.26 | GALNT2 | 2.30E−02 | 1.91E−01 | −1.51 |
| ENPP-7 | 6.23E−04 | 1.93E−02 | −1.90 | ENPP.2 | 2.35E−02 | 1.93E−01 | −1.08 |
| JAM.A | 7.44E−04 | 2.23E−02 | −1.24 | p53 | 2.38E−02 | 1.95E−01 | 1.52 |
| ANGPTL7 | 7.83E−04 | 2.27E−02 | −1.56 | Common.beta.Chain | 2.43E−02 | 1.95E−01 | −1.40 |
| EDIL3 | 1.03E−03 | 2.89E−02 | −1.59 | Ryk | 2.43E−02 | 1.95E−01 | −1.38 |
| ANGPTL3 | 1.16E−03 | 3.17E−02 | −1.30 | IGSF4B | 2.52E−02 | 2.00E−01 | −1.19 |
| HO.1 | 1.35E−03 | 3.56E−02 | −1.42 | CD6 | 2.55E−02 | 2.01E−01 | −7.69 |
| IGFBP-3 | 1.48E−03 | 3.81E−02 | 1.15 | PDGF-R-alpha | 2.58E−02 | 2.01E−01 | N/D |
| AFP | 1.69E−03 | 4.22E−02 | 2.32 | TPO | 2.76E−02 | 2.13E−01 | 1.31 |
| FGF-12 | 2.07E−03 | 5.02E−02 | −1.36 | DAN | 2.81E−02 | 2.15E−01 | 96.39 |
| CNTF | 2.13E−03 | 5.04E−02 | N/D | Cortactin | 2.83E−02 | 2.15E−01 | NA |
| Aminopeptidase.LRAP | 2.22E−03 | 5.10E−02 | −1.78 | ErbB4 | 2.85E−02 | 2.15E−01 | −1.91 |
| Semaphorin.7A | 2.49E−03 | 5.59E−02 | −1.39 | TGFb-RIII | 2.91E−02 | 2.18E−01 | −1.24 |
| Cystatin-SN | 2.62E−03 | 5.70E−02 | N/D | SorCS1 | 3.01E−02 | 2.22E−01 | −2.11 |
| IL-2 | 2.67E−03 | 5.70E−02 | −1.22 | GIF | 3.01E−02 | 2.22E−01 | −1.52 |
| IL-9 | 2.75E−03 | 5.70E−02 | −2.09 | Neudesin | 3.12E−02 | 2.26E−01 | −1.41 |
| CLEC10A | 2.80E−03 | 5.70E−02 | N/D | EphA1 | 3.12E−02 | 2.26E−01 | −1.54 |
| IL-1b | 2.85E−03 | 5.70E−02 | −4.10 | Glypican-1 | 3.17E−02 | 2.26E−01 | −1.25 |
| TACI | 2.99E−03 | 5.83E−02 | −1.53 | FAP | 3.17E−02 | 2.26E−01 | −1.09 |
| CA2 | 3.06E−03 | 5.84E−02 | 1.82 | JAM-C | 3.23E−02 | 2.28E−01 | 1.32 |
| Desmoglein-2 | 3.58E−03 | 6.42E−02 | −1.97 | SLAM | 3.29E−02 | 2.29E−01 | −1.56 |
| CD163 | 3.58E−03 | 6.42E−02 | −1.18 | ESAM | 3.29E−02 | 2.29E−01 | −1.18 |
| Nectin-2 | 3.58E−03 | 6.42E−02 | 1.17 | CHST2 | 3.34E−02 | 2.29E−01 | −1.77 |
| IGFBP-6 | 3.99E−03 | 7.03E−02 | −1.16 | MSP | 3.34E−02 | 2.29E−01 | −1.24 |
| CD200 | 4.27E−03 | 7.37E−02 | −1.18 | Draxin | 3.42E−02 | 2.31E−01 | −1.18 |
| Ret | 4.36E−03 | 7.39E−02 | 1.74 | Glypican-3 | 3.44E−02 | 2.31E−01 | −2.53 |
| Thrombospondin-2 | 4.53E−03 | 7.40E−02 | −2.36 | MOG | 3.45E−02 | 2.31E−01 | −2.37 |
| Tie-1 | 4.73E−03 | 7.40E−02 | −4.66 | OX40 | 3.49E−02 | 2.32E−01 | N/D |
| IL-27 | 4.76E−03 | 7.40E−02 | −1.88 | Trypsin-3 | 3.58E−02 | 2.36E−01 | −1.51 |
| CD99 | 4.76E−03 | 7.40E−02 | −1.10 | IL-4-Ra | 3.60E−02 | 2.36E−01 | Inf |
| HCC.4 | 4.86E−03 | 7.40E−02 | −1.12 | IL-17F | 3.64E−02 | 2.37E−01 | −1.50 |
| EphB4 | 4.86E−03 | 7.40E−02 | 1.21 | CLEC9a | 3.74E−02 | 2.42E−01 | −1.83 |
| Cystatin-E-M | 5.08E−03 | 7.60E−02 | −1.08 | TIMP-2 | 3.77E−02 | 2.42E−01 | −1.08 |
| bIG.H3 | 5.53E−03 | 8.15E−02 | −1.10 | RANTES | 3.90E−02 | 2.48E−01 | −1.05 |
| Testican-2 | 5.90E−03 | 8.51E−02 | −1.56 | FcERI | 3.97E−02 | 2.51E−01 | −1.48 |
| Epiregulin | 6.03E−03 | 8.51E−02 | −1.60 | ICOS | 4.10E−02 | 2.56E−01 | −1.17 |
| Syndecan-1 | 6.16E−03 | 8.51E−02 | −1.34 | CD49b | 4.10E−02 | 2.56E−01 | 1.21 |
| Fas | 6.16E−03 | 8.51E−02 | 1.31 | BMP.9 | 4.16E−02 | 2.57E−01 | −1.25 |
| ULBP-2 | 6.32E−03 | 8.60E−02 | NA | HS3ST4 | 4.23E−02 | 2.57E−01 | −1.27 |
| TSP-1 | 6.56E−03 | 8.79E−02 | −1.09 | Calreticulin | 4.24E−02 | 2.57E−01 | −1.95 |
| aFGF | 7.74E−03 | 1.02E−01 | −1.52 | TIMP-1 | 4.24E−02 | 2.57E−01 | −1.05 |
| Neuroligin-2 | 7.90E−03 | 1.03E−01 | −2.42 | TF | 4.31E−02 | 2.57E−01 | −1.73 |
| CILP.1 | 8.08E−03 | 1.04E−01 | −1.26 | VEGF-R3 | 4.31E−02 | 2.57E−01 | 1.21 |
| HAO.1 | 8.23E−03 | 1.04E−01 | 1.47 | DBH | 4.32E−02 | 2.57E−01 | −4.34 |
| FCAR | 8.42E−03 | 1.04E−01 | −1.26 | Nogo-A | 4.35E−02 | 2.57E−01 | −1.51 |
| EGF | 8.42E−03 | 1.04E−01 | 1.17 | Caspr2 | 4.53E−02 | 2.64E−01 | −1.48 |
| TFPI | 8.95E−03 | 1.09E−01 | −1.16 | IL-22 | 4.53E−02 | 2.64E−01 | 1.20 |
| proGRP | 9.32E−03 | 1.09E−01 | −1.35 | CLEC-1 | 4.56E−02 | 2.64E−01 | −4.74 |
| MIP.1d | 9.32E−03 | 1.09E−01 | −1.14 | TRAIL-R1 | 4.58E−02 | 2.64E−01 | −1.82 |
| FCRL2 | 9.51E−03 | 1.09E−01 | −1.11 | DSCAM | 4.69E−02 | 2.66E−01 | −1.44 |
| Pepsinogen-I | 9.51E−03 | 1.09E−01 | −1.10 | Kallikrein.14 | 4.69E−02 | 2.66E−01 | −1.11 |
| BMP-7 | 9.70E−03 | 1.09E−01 | −1.34 | BMP-2 | 4.99E−02 | 2.81E−01 | −1.26 |
| LAMP | 9.70E−03 | 1.09E−01 | −1.18 | Enteropeptidase | 5.00E−02 | 2.81E−01 | −1.31 |
| PSA.total | 1.03E−02 | 1.14E−01 | −1.28 | | | | |

TABLE 4
Biomarkers from Table 3 with FDR-adjusted
p-value < 0.05 (n = 36)

| | FDR adj | Fold Change | |
|---|---|---|---|
| Protein | p-value | (FC) | FC < [2] |
| IL-8 | 2.12E−07 | −6.48 | yes |
| GM-CSF | 1.87E−07 | −6.31 | yes |
| IL-17 | 1.26E−08 | −3.78 | yes |
| IL-10 | 4.77E−08 | −3.07 | yes |
| IL-1ra | 1.25E−02 | −2.94 | yes |
| IL-6 | 3.54E−09 | −2.85 | yes |
| IFNg | 4.86E−07 | −2.54 | yes |
| IL-12p70 | 1.26E−08 | −2.40 | yes |
| G-CSF | 1.25E−02 | −2.26 | yes |
| IL-1a | 1.68E−06 | −2.16 | yes |
| IL-15 | 6.63E−06 | −2.05 | yes |
| TNFa | 1.50E−07 | −1.95 | — |
| CF-XIV | 1.15E−02 | −1.92 | — |
| ENPP.7 | 1.93E−02 | −1.90 | — |
| TNFb | 1.68E−06 | −1.86 | — |
| SR.AI | 6.12E−03 | −1.84 | — |
| IL-13 | 2.69E−06 | −1.79 | — |
| IL-16 | 1.10E−04 | −1.78 | — |
| IL-4 | 8.42E−06 | −1.71 | — |
| BLC | 7.96E−03 | −1.67 | — |
| IL-5 | 5.78E−05 | −1.63 | — |
| IL-11 | 6.56E−06 | −1.60 | — |
| EDIL3 | 2.89E−02 | −1.59 | — |
| IL-7 | 1.67E−05 | −1.59 | — |
| ANGPTL7 | 2.27E−02 | −1.56 | — |
| HO-1 | 3.56E−02 | −1.42 | — |
| Fetuin-A | 1.41E−04 | −1.36 | — |
| FGF-12 | 5.02E−02 | −1.36 | — |
| ANGPTL3 | 3.17E−02 | −1.30 | — |
| JAM.A | 2.23E−02 | −1.24 | — |
| IL-1-R3 | 3.79E−03 | −1.21 | — |
| RBP4 | 6.63E−06 | −1.19 | — |
| IL-6R | 6.04E−03 | −1.12 | — |
| IGFBP-3 | 3.81E−02 | 1.15 | — |
| VEGF-R2 | 2.80E−03 | 1.26 | — |
| AFP | 4.22E−02 | 2.32 | yes |

As shown in Table 4, a subgroup of 12 biomarkers showed both ≥[2]-fold change (FC) in biomarker levels between TD and ASD groups (indicated as ‘yes’ in Table 4) and FDR-adjusted p-value<0.05. This subgroup of 12 biomarkers, listed also in Table 5, was retained for further analysis.

2.4 Selection of Highly Correlated Biomarkers

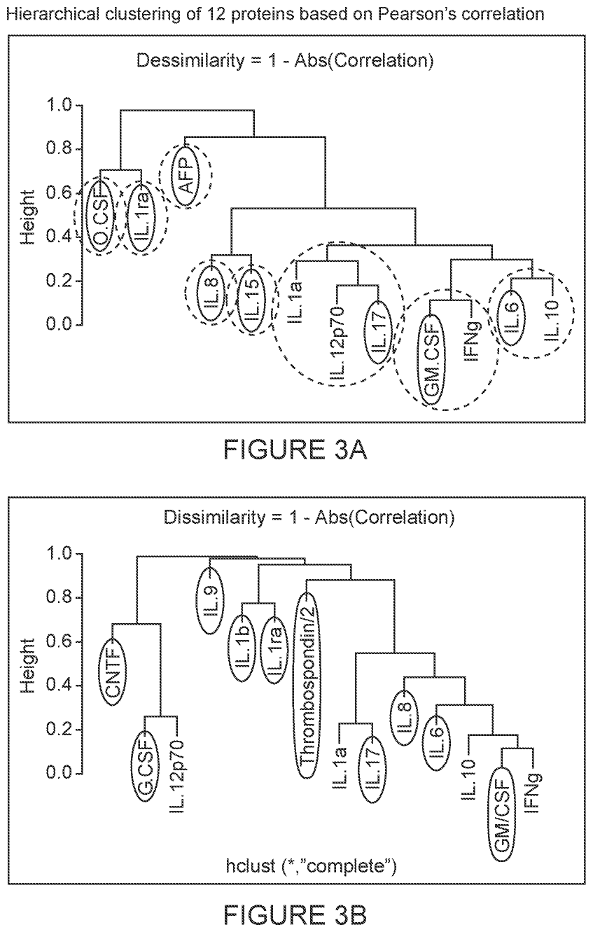

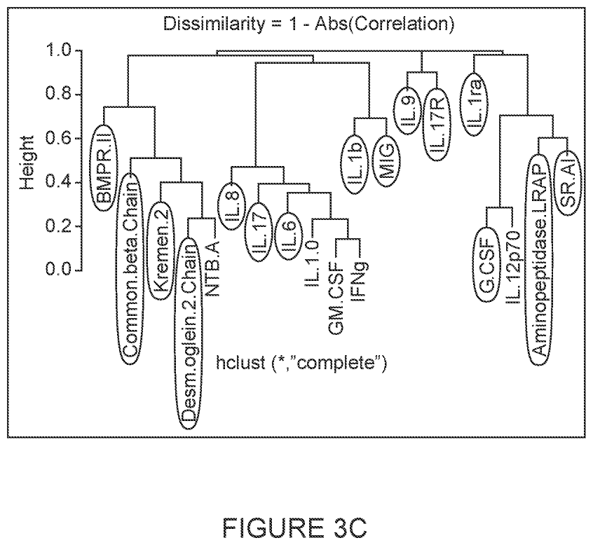

Many classification models, including logistic regression, are sensitive to dependent variables that are highly correlated (multi-colinearity). To address this issue, the Pearson's correlation coefficient (r) was calculated between any pairs of the 12 selected biomarkers. Table 5 represents the correlation matrix for the 12 selected biomarkers. These data were created as the basis for carrying out a hierarchical clustering for visualization purposes represented in FIG. 3A, where dashed circles denote protein groups and solid circles denote representative protein per group. The biomarkers were manually divided to groups, and a representative biomarker for each group was chosen. Note that a group can be represented in further analyses by only one biomarker.

The criteria for selecting the representative biomarkers were as follows:
(a) The biomarker correlated highly with the rest of the biomarkers in the group (r>0.7; underlined in Table 5).
(b) The biomarker did not correlate (r<0.7) with any of other representative biomarkers.

TABLE 5
Correlation matrix for 12 selected biomarkers

| | | | IL- | | | | | | | IL- | G- | |
|---|---|---|---|---|---|---|---|---|---|---|---|---|
| | IL-6 | IL-17 | 12p70 | IL-10 | GM-CSF | IL-8 | IFNg | IL-1a | IL-15 | 1ra | CSF | AFP |
| IL-6 | 1 | 0.69 | 0.8 | 0.78 | 0.7 | 0.61 | 0.75 | 0.66 | 0.59 | 0.14 | 0.28 | −0.34 |
| IL-17 | 0.69 | 1 | 0.82 | 0.71 | 0.67 | 0.58 | 0.68 | 0.7 | 0.55 | 0.12 | 0.3 | −0.26 |
| IL- | 0.8 | 0.82 | 1 | 0.75 | 0.69 | 0.57 | 0.73 | 0.79 | 0.6 | 0.15 | 0.32 | −0.23 |
| 12p70 | | | | | | | | | | | | |
| IL-10 | 0.78 | 0.71 | 0.75 | 1 | 0.75 | 0.68 | 0.75 | 0.64 | 0.72 | 0.14 | 0.34 | −0.31 |
| GM-CSF | 0.7 | 0.67 | 0.69 | 0.75 | 1 | 0.6 | 0.88 | 0.67 | 0.53 | 0.11 | 0.37 | −0.15 |
| IL-8 | 0.61 | 0.58 | 0.57 | 0.68 | 0.6 | 1 | 0.65 | 0.47 | 0.68 | 0.03 | 0.35 | −0.23 |
| IFNg | 0.75 | 0.68 | 0.73 | 0.75 | 0.88 | 0.65 | 1 | 0.78 | 0.58 | 0.1 | 0.34 | −0.2 |
| IL-1a | 0.66 | 0.7 | 0.79 | 0.64 | 0.67 | 0.47 | 0.78 | 1 | 0.49 | 0.15 | 0.31 | −0.2 |
| IL-15 | 0.59 | 0.55 | 0.6 | 0.72 | 0.53 | 0.68 | 0.58 | 0.49 | 1 | −0.02 | 0.19 | −0.28 |
| IL-1ra | 0.14 | 0.12 | 0.15 | 0.14 | 0.11 | 0.03 | 0.1 | 0.15 | −0.02 | 1 | 0.3 | −0.08 |
| G-CSF | 0.28 | 0.3 | 0.32 | 0.34 | 0.37 | 0.35 | 0.34 | 0.31 | 0.19 | 0.3 | 1 | −0.09 |
| AFP | −0.34 | −0.26 | −0.23 | −0.31 | −0.15 | −0.23 | −0.2 | −0.2 | −0.28 | −0.08 | −0.09 | 1 |

Following the aforementioned feature selection, a subgroup of 8 biomarkers were retained for further analysis: GM-CSF, IL1ra, AFP, IL-8, IL-15, IL-17, G-CSF, IL-6.

2.5 Univariate Logistic Regression

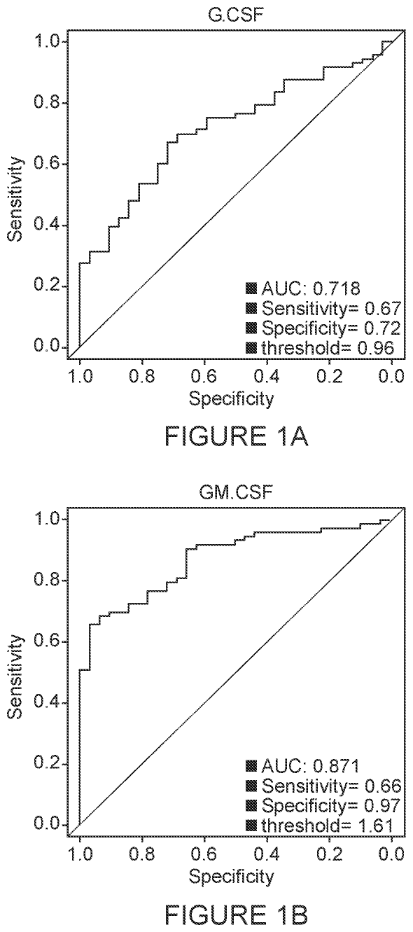

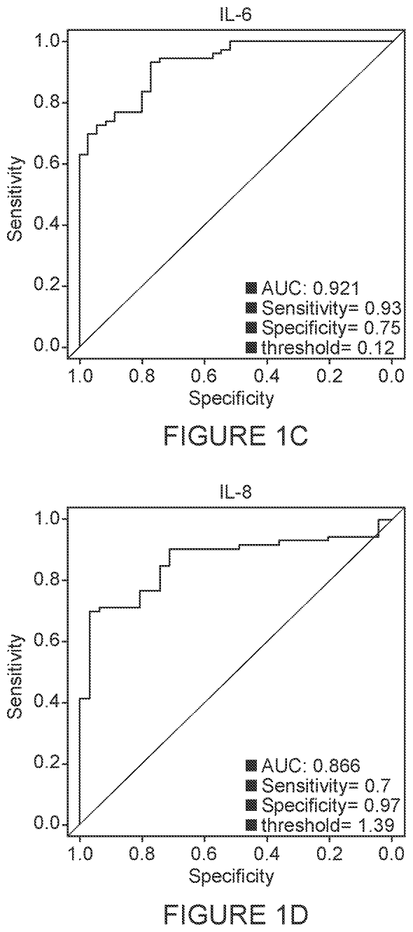

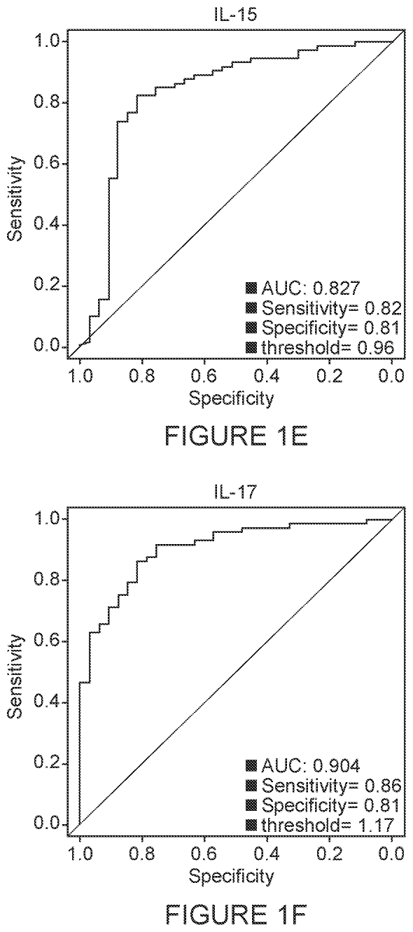

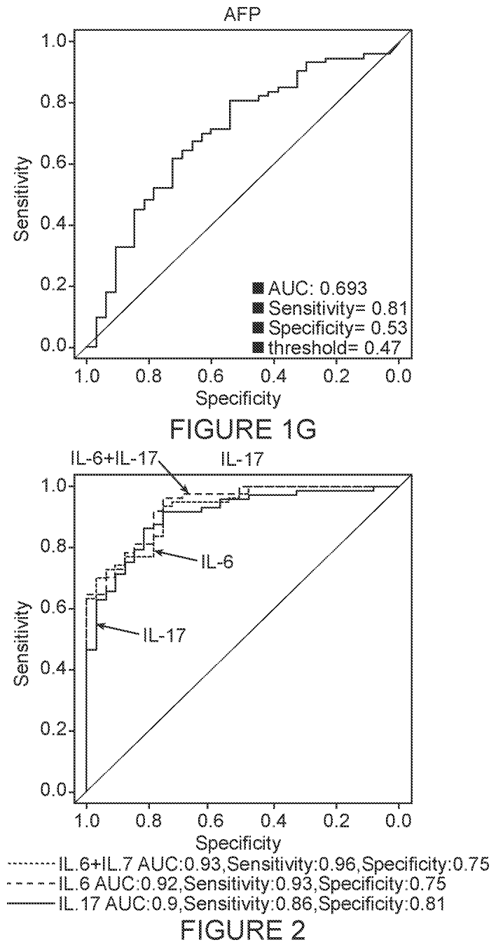

The aforementioned subgroup of 8 selected proteins underwent a univariate logistic regression test, in which 7 proteins had a significant p-value (<0.05: G-CSF, GM-CSF, IL-6, IL-8, IL-15, IL-17 and AFP. A ROC curve was calculated for each of the 7 biomarkers by plotting the true positive rate (sensitivity) against the false positive rate (1 - specificity) at various threshold settings (FIGS. 1A-1G for G-CSF, GM-CSF, IL-6, IL-8, IL-15, IL-17 and AFP, respectively). This analysis illustrated the diagnostic ability of a binary classifier system as its discrimination threshold was varied. In each ROC graph, the following parameters are indicated: AUC; Youden index threshold of the regression model; sensitivity and specificity at the Youden threshold.

2.6 Multivariate Analysis

2.6.1 Analysis Using the Training Subset

To select biomarkers for the multivariate logistic regression model, a stepwise logistic regression was performed. In this stepwise algorithm, biomarkers were successively removed or added in order to obtain a model with the smallest Akaike information criterion (AIC) value. AIC is an estimator of the relative quality of statistical models for a given dataset and was used for comparing between models. As a result of this analysis, a multivariate model with IL-6 and IL-17 biomarkers yielded the lowest AIC value, and therefore these two (2) biomarkers were selected (out of the initial 7 biomarkers) for constructing the multivariate logistic regression model. Table 6 represents the coefficient univariate logistic regression model of each of these two biomarkers alone, and the multivariate model using both biomarkers.

TABLE 6
Coefficient univariate logistic regression

| | | | Std. | z | |
|---|---|---|---|---|---|
| Model | | Estimate | Error | value | Pr(>\|z\|) |
| Y~IL-6 + | (Intercept) | 4.701267 | 0.861106 | 5.460 | 4.77e−08 *** |
| IL-17 | IL-6 | −0.03584 | 0.009766 | −3.670 | 0.000243 *** |
| | IL-17 | −0.02961 | 0.012551 | −2.359 | 0.018333 * |
| Y~IL-6 | (Intercept) | 4.602017 | 0.870455 | 5.287 | 1.24e−07 *** |
| | IL-6 | −0.04686 | 0.009904 | −4.731 | 2.23E−06 *** |
| Y~IL-17 | (Intercept) | 2.85968 | 0.49389 | 5.79 | 7.03E−09 *** |
| | IL-17 | −0.06437 | 0.01396 | −4.609 | 4.04E−06 *** |

Significance codes: *** = 0;
** = 0.001;
* = 0.01

Table 7 represents the AUC, sensitivity and specificity for the multivariate model and for each biomarker alone, under the threshold where Youden index is maximal. As shown in this Table, the multivariate model yielded better sensitivity and specificity than the models built with each biomarker alone, hence providing superior discrimination between groups.

TABLE 7
Performance results for univariate and multivariate analyses

| Model | AUC | Sensitivity | Specificity | Threshold |
|---|---|---|---|---|
| Y~IL-6 + IL-17 | 0.93 | 0.96 | 0.75 | 0.07 |
| Y~IL-6 | 0.92 | 0.93 | 0.75 | 0.12 |
| Y~IL-17 | 0.9 | 0.86 | 0.81 | 0.17 |

ROC curves were plotted for each of the univariate models as well as for the multivariate model (FIG. 2). An exemplary logistic regression formula as obtained for the multivariate model to predict whether a given sample belongs to the TD or ASD group is as follows:
Yi=4.7−0.036*IL-6−0.03*IL-17 for threshold of 0.072
   wherein ASD>0.072 and TD<0.072 and wherein for each marker in the formula the amount, or relative amount, of the marker is used to calculate a numerical value (Yi). A result that is higher than a predetermined threshold indicates that the sample is ASD, viz., derived from a subject having ASD or susceptible to ASD.

2.6.2 Testing the Multivariate Model on the Testing Subset

The performance of the multivariate model was tested on the testing subset, which represents an independent set of samples according to the following method. The expression level values of IL-6 and IL-17 in each of the samples of the testing subset were assigned into the logistic regression formula represented above, and the Youden index threshold from the training set (0.072) was used to predict for each individual sample whether it is TD or ASD. The results are shown in Table 8. The results yielded sensitivity of 0.90 and specificity of 0.53, which, compared to the performance obtained with the training set, has a similar sensitivity and significant decrease in specificity.

TABLE 8
Summary performance of logistic models generated
using the training subset and the testing subset

| Biomarkers | Training set | | Testing set | | |
|---|---|---|---|---|---|
| chosen by | Performance | | Performance | | |
| stepwise | Sensi- | Speci- | Sensi- | Speci- | |
| analyses | tivity | ficity | tivity | ficity | Threshold |
| IL-6, IL-17 | 96 | 75 | 90 | 53 | 0.072 |

2.6.3 Repeating the Analyses with Two Additional Random Data Splits to Training and Testing Subsets

To check the consistency of the findings, the process of data analysis described above was repeated two more times (repetitions A & B), each time starting with a different split of the database into training and testing subsets. In each split, 70% of the samples were randomly selected for the training set and the remaining 30% of samples were allocated to the testing subset. As in the first analysis, the proportion between TD (32%) and ASD (68%) was preserved.

2.6.3.1 Repetition Test A and B—Biomarker Selection for Multivariate Analysis

The new training sets (A and B) were analyzed as described above, resulting in 14 selected biomarkers for training set A and 20 selected biomarkers for set B (detailed in Table 9). These biomarkers had a significant difference in levels between the ASD and TD groups with FDR-adjusted p-value<0.05 and at least a 2-fold difference between groups.

TABLE 9
Biomarkers in the two (2) training subsets-Repetitions A and B

| | Repetition A | | | Repetition B | |
|---|---|---|---|---|---|
| | | Multivariate | | | Multivariate |
| No. | Biomarker | Analysis | No. | Biomarker | Analysis |
| 1 | CNTF | yes | 1 | BMPR-II | yes |
| 2 | G-CSF | yes | 2 | Common- | yes |
| 3 | IL-12p70 | | | beta-chain | |
| 4 | IL-9 | yes | 3 | Kremen-2 | yes |
| 5 | IL-1b | yes | 4 | Desmoglein-2 | yes |
| 6 | IL-1ra | yes | 5 | NTB-A | |
| 7 | Thrombo- | yes | 6 | IL-8 | yes |
| | spondin-2 | | 7 | IL-17 | yes |
| 8 | IL-1a | | 8 | IL-6 | yes |
| 9 | IL-17 | yes | 9 | IL-10 | |
| 10 | IL-8 | yes | 10 | GM-CSF | |
| 11 | IL-6 | yes | 11 | IFNg | |
| 12 | IL-10 | | 12 | IL-1b | yes |
| 13 | GM-CSF | yes | 13 | MIG | yes |
| 14 | IFNg | | 14 | IL-9 | yes |
| | | | 15 | IL-17R | yes |
| | | | 16 | IL-1ra | yes |
| | | | 17 | G-CSF | yes |
| | | | 18 | IL-12p70 | |
| | | | 19 | Amino- | yes |
| | | | | peptidase- | |
| | | | | LRAP | |
| | | | 20 | SR-A1 | yes |

In the next step, these biomarkers were subjected to selection based on Pearson's correlation (r<0.7) and hierarchical clustering as represented in FIGS. 3A and 3B. For repetition A (FIG. 3A) 10 biomarkers were selected (CNTNF, G-CSF, IL-9, IL-1b, IL-1RA, Thrombospondin-2, IL-17, IL-8, IL-6 and GM-CSF) and for repetition B (FIG. 3B) 15 biomarkers were selected for the multivariate analyses, seven (7) of which are common with repetition A: G-CSF, IL-9, IL-1b, IL-1RA, IL-17, IL-8, IL-6, and the additional eight (8) biomarkers are as follows: BMPR-II, Common-beta-chain, Kremen-2, Desmoglein-2, MIG, IL-17R, Aminopeptidase-LRAP and SR-A1.

2.6.3.2 Multivariate Analyses of Repetitions A and B

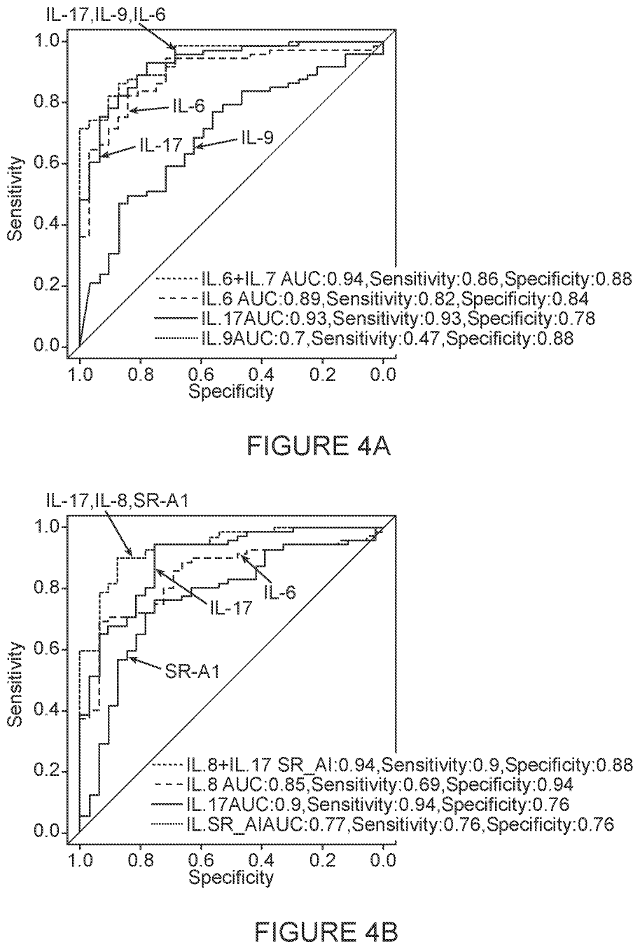

Analysis was performed on the selected biomarkers indicated by ‘yes’ in Table 9. As a result of this analysis, IL-6, IL-17 and IL-9 biomarkers were selected out of the initial 10 biomarkers for showing best performance in differentiating between TDs and ASDs, in order to construct the multivariate logistic regression model (Table 10; under the threshold where Youden index is maximal). For repetition B, IL-8, IL-17 and SR-AI biomarkers were selected out of the initial 15 biomarkers as the outcome of the MLR model (Table 10). The data in Table 10 for each biomarker alone show the advantage of a multivariate model in that they generate a more balanced performance. FIGS. 4A and 4B represent the ROC and performance values obtained for each of repetition analysis A and B, respectively.

TABLE 10
Performance results for Univariate and multivariate
analyses for Repetitions A and B.

| Repetitions A | | | | Repetitions B | | | |
|---|---|---|---|---|---|---|---|
| Biomarker | AUC | Sensitivity | Specificity | Biomarker | AUC | Sensitivity | Specificity |
| Y~IL-6 + | 0.94 | 0.86 | 0.88 | Y~SR-AI + | 0.94 | 0.9 | 0.88 |
| IL-17 + IL-9 | | | | IL-17 + IL-8 | | | |
| Y~IL-6 | 0.89 | 0.82 | 0.84 | Y~IL-17 | 0.9 | 0.94 | 0.76 |
| Y~IL-17 | 0.93 | 0.93 | 0.78 | Y~IL-8 | 0.85 | 0.69 | 0.94 |
| Y~IL-9 | 0.7 | 0.47 | 0.88 | Y~SR-AI | 0.77 | 0.76 | 0.76 |

The logistic regression models generated multivariate analyses for repetitions A & B selected biomarkers are presented in Table 11.

TABLE 11
Summary of exemplary logistic regression
model equations (logit) generated using two
additional random split to training set (70%) and test sets (30%)

| Repetition | Biomarkers | Formula | Threshold |
|---|---|---|---|
| A | IL-6, IL-17, | Logit = 5 − 0.012*IL-6 − | 1064 |
| | IL-9 | 0.0885*IL-17 − 0.0005*IL-9 | |
| B | SR-AI, | Logit = 4.76 + 0.015*IL-8 − | 1176 |
| | IL-17, IL-8 | 0.1*IL-17 − 0.001*SR--AI | |

2.6.3.3 Testing the Repetitions A and B Multivariate Model on the Testing Subsets

The performances of the multivariate models were tested on the testing subsets, which represent an independent set of samples according to the method described in this example. The biomarker expression values of each biomarker in each of the samples of the testing subset were assigned into the logistic regression model equations represented in Table 11, and the Youden index threshold from the training set (Repetition A, cut off: 1.064; Repetition B, cut off: 1.176) was used to predict whether each individual sample is in the TD or ASD group, as shown in Tables 11 and 12.

TABLE 12
Summary performance of the exemplary logistic models shown in Table 11, generated
using the training subset and the testing subset for Repetition A & B

| | | Training subset | | Testing subset | | |
|---|---|---|---|---|---|---|
| Repetition | Biomarkers chosen by | Performance | | Performance | | |
| No. | stepwise analyses | Sensitivity | Specificity | Sensitivity | Specificity | Threshold |
| A | IL-6, IL-17, IL-9 | 86 | 88 | 79 | 80 | 1.064 |
| B | SR-AI, IL-17, IL-8 | 90 | 88 | 73 | 93 | 1.176 |

For repetition A, the validation revealed a drop of sensitivity from 86% to 79% and drop of specificity from 88% to 80%. For repetition B, the validation revealed a drop of sensitivity from 90% to 73% and increase of specificity from 88% to 93%.

### Example 3

### Identification of Biomarker Combinations for ASD/TD Prediction Using K-Fold Cross-Validation Approach

This study was using the “expanded database” which was composed of 102 ASD samples (54% of total) and 97 TD samples (48% of total). In this study, 5 (denoted: A-E) processes or methods were applied.

A. Division into training/testing sets and Feature Selection—A standard 10-fold cross-validation procedure was applied, using a sampling method that aims to minimize the difference in ASD/TD ratio between the folds. Data in the biomarker level database generated for 102 ASD samples and 97 TD samples were divided into 10 sets while keeping the ASD/TD ratio in each set. This step resulted in assigning each case into one of 10 “folds”, with 19-20 subjects in each fold.

For each fold, one set was held out as a testing set, and the remaining 90% were used for training. This resulted in 10 “folds”. The training set (90% of the data) of each fold was subjected to feature selection (MLR). With this procedure, training for each set was conducted with somewhat overlapping datasets, but the test set was unique for each fold; every case was used in the testing in one and only fold.

B. Cleaning the biomarker-feature levels database from “mostly zero” features—For each of the training-set folds, a feature was eliminated from subsequent analyses if P1=0.6 or more of the feature values in ASD cases and in TD cases was zero. The number of features eliminated in each fold with this step was 8.57±2.95 biomarkers in average as represented in column F(P1) in Table 13. Specifically, Table 13 shows for each model the Accuracy, Sensitivity, Specificity and the F1-Score statistics. This also shows the number of features remaining after ‘mostly zero’ filtering, denoted as F(P1) and after also filtering by feature correlation clustering, denoted as F(P2). The summary statistics for the 10-fold cross-validation is also shown in Table 13, denoted ‘MLR total’, providing the mean±standard deviation for the 10 folds.

TABLE 13
Performance of MLR models

| | F(P1) | F(P2) | Accuracy | Sensitivity | Specificity | F1 score |
|---|---|---|---|---|---|---|
| MLR1 | 89 | 105 | 0.800 | 0.818 | 0.778 | 0.818 |
| MLR2 | 82 | 107 | 0.750 | 0.909 | 0.556 | 0.800 |
| MLR3 | 85 | 101 | 0.700 | 0.800 | 0.600 | 0.727 |
| MLR4 | 86 | 107 | 0.950 | 1.000 | 0.900 | 0.952 |
| MLR5 | 87 | 109 | 0.850 | 0.900 | 0.800 | 0.857 |
| MLR6 | 82 | 107 | 0.800 | 0.900 | 0.700 | 0.818 |
| MLR7 | 88 | 109 | 0.900 | 0.900 | 0.900 | 0.900 |
| MLR8 | 82 | 108 | 0.850 | 0.900 | 0.800 | 0.857 |
| MLR9 | 86 | 114 | 0.700 | 0.700 | 0.700 | 0.700 |
| MLR10 | 90 | 103 | 0.947 | 0.900 | 1.000 | 0.947 |
| MLR | 85.70 +/− 2.95 | 107.00 +/− 3.56 | 0.82 +/− 0.09 | 0.87 +/− 0.08 | 0.77 +/− 0.14 | 0.84 +/− 0.08 |
| total | | | | | | |

C. Feature clustering by correlation — In this step, which is performed only on the training data, any two features with Spearman correlation coefficient (R²) of P2 or more were clustered together. By default, a value of P2=0.5 was used. Clustering was agglomerative, i.e., if a feature was correlated with any member of a cluster, this feature (and any feature clustered thereto) was added to that cluster. For every correlation cluster, one representative feature with the highest mean correlation to all other features in the same cluster was chosen and all other features were eliminated. The number of features remaining after clustering is represented in column F(P2) of Table 13.

D. MLR. For each fold, features (proteins) were ranked by their ability to perform as a single marker for ASD using the sklearn.feature_selection.f_regression function. This function assigns to each feature the number of times it is found in the root of ASD/TD prediction trees. In other words, it quantifies the frequency with which each feature is used as the first split in discriminating between TD and ASD cases. After ranking, the top 1% of features (about 7-8 protein biomarkers) were chosen for the MLR model. The features selected for each fold are always included in the MLR model and thus as described in the MLR equations provided below, including in Table 15; the algorithm seeks for these features the coefficients that give the best separation between ASD and TD cases. The MLR method considered all (first and second) the database cases for the analysis with no option for undetermined cases.

In the 10-fold cross-validation procedure that was applied for MLR models, 90% of the data were used for training every time/cycle and 10% for testing, replacing the cases used for testing 10 times and thus getting 10 decision trees and 10 performance statistics.

E. General remark on evaluating the results—Since a 10-fold cross-validation approach was applied, every value (accuracy, sensitivity, etc.) was calculated 10 times. Thus, the reported mean and standard variation of each measure of success is as calculated in the test data of each fold. Therefore, it is important to note that standard variation is probably under-estimated, since the changes in the data may have been under-estimated due to the large overlap between the training sets.

The results of this study, obtained following application of the above listed statistical approaches, are summarized in Tables 14 and 15. (i) As summarized in Table 13, MLR models gave altogether an average accuracy of 10-fold cross-validation of 82±9%. (ii) The quality and features of MLR models. Table 14 provides a list of the features (proteins) used to construct the MLR models which are relatively stable: IFNγ, IL-10, IL-17, TNF-α and aFGF occur in all 10 models, IL-4Ra, IL-6, and IL-1a, occur in over half of the MLR models, procalcitonin occurs in 4 of 10 models, TFPI and TCPTP occur in 3, RBP4 and Kallikrein_1 occur in 2 and semaphoring 7A, carboxypeptidase_A2 and LIGHT occur in 1 (Tables 14 and 15).

TABLE 14
Features used to construct MLR models, for each
feature the number of folds in which this feature
was chosen is indicated, where features recurring more than once
are in bold and features recurring also in Example 2 are underlined.

| | Recurrence in No. | Recurrence in No. |
|---|---|---|
| | of folds | of folds |
| Marker | (Example 3) | (Example 2) |
| IL-17 | 10 | 1 |
| aFGF | 10 | 0 |
| IFNg | 10 | 0 |
| IL-10 | 10 | 0 |
| TNFa | 10 | 0 |
| IL-4Ra | 9 | 0 |
| IL-6 | 8 | 1 |
| IL-1a | 6 | 0 |
| Procalcitonin | 4 | 0 |
| TC_PTP | 3 | 0 |
| TFPI | 3 | 0 |
| RBP4 | 2 | 0 |
| Kallikrein_1 | 2 | 0 |
| Carboxypeptidase_A2 | 1 | 0 |
| LIGHT | 1 | 0 |
| Semaphorin_7A | 1 | 0 |
| IL-8 | 0 | 1 |
| IL-9 | 0 | 1 |
| SR-AI | 0 | 1 |

(iii) The MLR Decision Trees (Models)

The 10 exemplary MLR equations obtained with 10-fold cross validation process are represented in Table 15. Each equation can be used to predict the ASD or TD status of the case as follows: the raw marker measurements are z-normalized for each biomarker (i.e., the mean value for each biomarker is subtracted from each measurement, and the result is divided by the standard deviation); upon inserting the normalized values of each marker in the equation, the prediction is ASD if the result is positive, and the prediction is TD if the result is negative.

TABLE 15
MLR Equations

| No. | Equation |
|---|---|
| 1 | −0.13*IFNg − 1.25*IL10 − 0.84*IL17 + 0.27*TNFa − 1.70*aFGF + |
| | 1.09*IL4Ra − 1.08*IL6 − 0.31*IL1a − 0.66*RBP4 − 0.33 |
| 2 | −0.22*IFNg − 1.09*IL10 − 0.96*IL17 + 0.32*TNFa − 1.76*aFGF + |
| | 1.42*IL4Ra − 1.07*IL6 − 0.11*IL1a − 0.48*TFPI − 0.34 |
| 3 | −0.23*IFNg − 1.15*IL10 − 1.03*IL17 + 0.33*TNFa − 1.59*aFGF + |
| | 1.08*IL4Ra − 0.87*IL6 − 0.11*IL1a − 0.56*TFPI − 0.34 |
| 4 | −0.08*IFNg − 1.00*IL10 − 1.03*IL17 − 0.13*TNFa − 1.54*aFGF + |
| | 1.26*IL4Ra − 0.81*IL6 − 0.24*IL1a − 0.93*Kallikrein_1 − 0.49 |
| 5 | +0.17*IFNg − 0.69*IL10 − 1.16*IL17 − 0.04*TNFa − 2.14*aFGF − |
| | 1.07*LIGHT − 0.51*IL6 − 0.45*IL1a − 0.67*Semaphorin7A − 0.68 |
| 6 | −0.16*IFNg − 1.19*IL10 − 0.93*IL17 + 0.11*TNFa − 1.44*aFGF + |
| | 0.92*IL4Ra − 1.14*IL6 + 1.43*Procalcitonin − 0.48*TFPI − 0.22 |
| 7 | −0.02*IFNg − 1.06*IL10 − 0.98*IL17 + 0.26*TNFa − 1.14*aFGF + |
| | 0.84*IL4Ra − 1.35*IL6 + 1.27*Procalcitonin − 0.66*TCPTP − 0.17 |
| 8 | −0.07*IFNg − 0.92*IL10 − 0.89*IL17 − 0.15*TNFa − 1.17*aFGF + |
| | 0.80*IL4Ra − 1.39*IL6 + 1.44*Procalcitonin − 0.81*TCPTP − 0.26 |
| 9 | −0.36*IFNg − 1.25*IL10 − 1.44*IL17 + 0.12*TNFa − 1.35*aFGF + |
| | 0.85*IL4Ra − 1.05*Carboxypeptidase_A2 + 1.58*Procalcitonin − |
| | 0.58*Kallikrein_1 − 0.28 |
| 10 | −0.06*IFNg − 1.63*IL10 − 1.09*IL17 + 0.32*TNFa − 1.58*aFGF + |
| | 0.79*IL4Ra − 0.52*RBP4 − 0.52*IL1a − 0.92*TCPTP − 0.47 |

Each of the MLR equations listed in Table 15 can be used to predict the ASD or TD status of the case as follows: first, the raw marker measurements are z-normalized for each biomarker (i.e., the mean value for each biomarker is subtracted from each measurement, and the result is divided by the standard deviation). The normalized values of each marker are used in the equation. For each equation presented in Table 15, the threshold P is calculated as follows:

P=exp(Yi)/(1+exp(Yi)

where Yi=the result of an MLR equation (containing the expression values for each biomarker, as measure from a biological sample of a subject, exp =exponential, wherein when P>0.5 the subject is predicted as ASD and when P<0.5 the subject is predicted to be TD.

As explained above, the datasets between the 10 folds were only somewhat overlapping datasets, but the test set was unique for each fold—every case was used in the testing in one and only fold. Thus, the fact that the MLR equations exhibit several dominant biomarkers, namely, biomarkers having a coefficient equal or higher than 0.8, indicates that a panel of biomarkers including these biomarkers provides a strong tool for diagnosing ASD. The dominant biomarkers presented throughout the MLR equations are: IL-17, aFGF and IL-10. The biomarkers IL-4RA and IL-6 were also dominant in most of the MLR equations. Thus, the analysis revels the significance of IL-17, aFGF, IL-10, IL-4RA and IL-6 in characterizing and identifying ASD.

Of note, IL-17, IL-10 and IL-6 were shown to be dominant in both analyses (Examples 2 and 3) indicating that a panel comprising these three biomarkers can reliably detect ASD and distinguish ASD from TD.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A kit for identifying a subject having autism spectrum disorders (ASD) or susceptibility to ASD, the kit comprising: (a) means for measuring the levels of a plurality of biomarker proteins selected from Tables 3, 4, 5, 6, 9 or 14 in a biological sample obtained from a subject; (b) a predetermined logistic regression model equation and a cutoff value for the plurality of biomarkers; and (c) means for obtaining a numerical value for the predetermined logistic regression model equation for the measured levels of the plurality of biomarker proteins, wherein a numerical value above said cutoff value identifies said subject as having ASD or susceptibility to ASD.

2. The kit according to claim 1, wherein the plurality of biomarker proteins is selected from Table 5, Table 9 or Table 14.

3. (canceled)

4. (canceled)

5. The kit according to claim 1, wherein the plurality of protein biomarkers comprises IL-17.

6. The kit according to claim 5, wherein the plurality of protein biomarkers further comprises at least one protein selected from the group consisting of: IL-6, IL-8, IL-9, IL-10, G-CSF and GM-CSF.

7. The kit according to claim 1, wherein the plurality of biomarker proteins comprises at least three biomarker proteins.

8. The kit according to claim 7, wherein the at least three biomarker proteins comprise IL-6, IL-10 and IL-17.

9. (canceled)

10. A method for diagnosing ASD or susceptibility to ASD, the method comprising:
(a) providing, for a plurality of biomarkers, a predetermined logistic regression model equation and a cutoff value, wherein the plurality of biomarker proteins is selected from Tables 3, 4, 5, 6, 9 or 14;
(b) determining, in a biological sample obtained from a subject, the level of each protein biomarker in the plurality of biomarker proteins; and
(c) incorporating the level of each protein biomarker in the predetermined logistic regression model equation, thereby obtaining a numerical value,
wherein a numerical value above said cutoff value identifies said subject as having ASD or susceptibility to ASD.

11. The method according to claim 10, wherein the plurality of biomarker proteins is selected from Table 5 Table 9 or Table 14.

12. (canceled)

13. (canceled)

14. The method according to claim 10, wherein the plurality of protein biomarkers comprises IL-17.

15. The method according to claim 14, wherein the plurality of protein biomarkers further comprises at least one protein selected from the group consisting of: IL-6, IL-8, IL-9, IL-10, G-CSF and GM-CSF.

16. The method according to claim 10, wherein the plurality of biomarker proteins comprises at least three biomarker proteins.

17. (canceled)

18. The method according to claim 16, wherein the at least three biomarker proteins comprise IL-6, IL-10 and IL-17.

19. The method according to claim 10, wherein the biological sample is derived from a subject of age between 1 year and 15 years.

20. A method for characterizing ASD or susceptibility to ASD, in a biological sample, the method comprising:
(a) obtaining a first group of biological samples from ASD subjects and a second group of biological samples from autism spectrum disorders (TD) subjects;
(b) selecting a first set of proteins in said first group and a second set of proteins in said second group, wherein each of said first and second sets comprises a plurality of proteins;
(c) dividing the proteins in the first set into a first training subset and a first testing subset and the proteins in the second set into a second training subset and a second testing subset, wherein training subset:testing subset ratio in each of said first and second sets corresponds to first group:second group ratio;
(d) comparing the level of each protein in the first training subset to the level of said each protein in the second training subset and identifying protein biomarkers whose level in the first training subset is significantly different from its level in the second training subset; and
(e) selecting a plurality of protein biomarkers that are highly correlated and have lowest AIC value for constructing a multivariate logistic regression model, said plurality of protein biomarkers characterizes ASD, or susceptibility to ASD.

21. The method according to claim 20, wherein the level of significance is calculated using Mann-Whitney test.

22. The method according to claim 20, further comprises selecting a plurality of protein biomarkers having FDR-adjusted p-value<0.05 and FC>2, prior to step (e).

23. The method according to claim 20, wherein said dividing is randomly dividing.

24. (canceled)

25. (canceled)

26. The method according to claim 20, wherein the biological sample is derived from a subject of age between 1 year and 15 years.

27. (canceled)

28. The method according to claim 20, wherein the reference value corresponds to the level of said the plurality of protein biomarkers in biological samples derived from a population of TD subjects.

29. The method according to claim 20, wherein the plurality of protein biomarkers is selected from the group consisting of the protein biomarkers listed in Table 4.

30.-34. (canceled)
